(19) United States
(12) Patent Application Publication
(10) Pub. No.: US 2025/0293873 A1
(43) Pub. Date: Sep. 18, 2025

(54) GENOMIC CRYPTOGRAPHY

(71) Applicants: THE BROAD INSTITUTE, INC., Cambridge, MA (US); PRESIDENT AND FELLOWS OF HARVARD COLLEGE, Cambridge, MA (US)

(72) Inventors: VERENA VOLF, Cambridge, MA (US); FEI CHEN, Cambridge, MA (US); SIMON ZHANG, Cambridge, MA (US)

(73) Assignees: THE BROAD INSTITUTE, INC., Cambridge, MA (US); PRESIDENT AND FELLOWS OF HARVARD COLLEGE, Cambridge, MA (US)

(21) Appl. No.: 19/224,661

(22) Filed: May 30, 2025

Related U.S. Application Data
(63) Continuation of application No. PCT/US2023/082038, filed on Dec. 1, 2023.
(60) Provisional application No. 63/429,359, filed on Dec. 1, 2022.

(51) Int. Cl. H04L 9/08; G16B 20/20; G16B 30/00; H04L 9/32

(52) U.S. Cl. CPC H04L 9/0866; G16B 20/20; G16B 30/00; H04L 9/3231

(57) ABSTRACT

Methods, systems, and compositions comprising genomic encryption are detailed herein. Also provided are methods and applications for authenticating biological material utilizing genomic encryption.

### CROSS-REFERENCE TO RELATED APPLICATIONS

This application claims the benefit of U.S. Provisional Application No. 63/429,359, filed Dec. 1, 2022. The entire contents of the above-identified applications are hereby fully incorporated herein by reference.

### TECHNICAL FIELD

The subject matter disclosed herein is generally directed to methods and systems of biological encoding, encryption, and authentication.

### BACKGROUND

DNA is the information storage medium of life. In recent years, there has been significant interest in leveraging DNA as a storage medium for digital (Church, Gao, and Kosuri 2012; Shipman et al. 2017; Yim et al. 2021) and biological data (Kalhor et al. 2018; McKenna et al. 2016; Tang and Liu 2018; Farzadfard et al. 2019). However, mechanisms of securing information in DNA from adversarial attack remain to be addressed.

Here, Applicants present an encryption scheme implemented in the DNA of living cells, that is based solely on the properties of DNA sequence analysis. Modern cryptography uses one or more keys to encode and decode information in order to guarantee message confidentiality between a sender and a receiver. Due to an exponential drop in sequencing cost (20 million-fold since 2004) and advances in genome engineering, information encoding in DNA has gained increased interest. The scheme presented here achieves information confidentiality and authentication for information encoded in DNA. Its applications include falsification-proof signatures of genetically modified organisms (including but not limited to cell lines, animals, and crops), which are passed on over generations and would allow an intended recipient to verify a strain while the installed signature would be invisible to others. Similarly, genomically barcoded biological materials have previously been demonstrated for supply chain validation (Qian et al. 2020), however, the genomic modifications proposed have been unencrypted and easy to detect, and vulnerable to falsification.

While information can be encrypted using existing algorithms prior to its encoding in DNA, a growing category of data is generated in vivo never passing through a conventional computer (Kalhor et al. 2018; McKenna et al. 2016; Tang and Liu 2018; Farzadfard et al. 2019; Choi et al. 2022) and genomic encryption schemes based solely on biological properties have to Applicants' knowledge not been reported. Prior work has demonstrated a double steganography approach in which a short synthesized DNA sequence is hidden in fragmented extracted DNA, which is then further concealed in a microdot (Clelland, Risca, and Bancroft 1999). However, while this approach provides security through obscurity, once a microdot is discovered and the DNA is sequenced, short strings of sequence that do not map to a reference genome can be easily detected. While previous schemes relied on the difficulty of DNA sequencing, here, Applicants assume DNA sequencing is abundant but instead relies on asymmetric sequencing cost due to encryption. The approach presented here provides security even if the cryptographic scheme is completely known, analogous to asymmetric computational costs in cryptographic hashes. Importantly, Applicants adhere to Shannon's Maxim (Shannon 1949) wherein the adversary has full knowledge of the encryption scheme barring the keys.

Applicants propose an encryption scheme, Genomic Sequence Encryption (GSE), which relies on the highly asymmetric cost of detecting mutations when the position of potentially edited bases is known versus if the whole genome has to be searched for mutated bases. If the key, i.e. genome coordinates at which an edit can be installed are known, targeted sequencing can be efficiently performed at high coverage. However, if the coordinates are unknown, all bases of the genome need to be analyzed, which is difficult not only due to the large genome sizes, but also due to errors in the process of detecting point mutations, and inherent mutational processes within cells. The difficulty of detecting low-frequency mutations and distinguishing between true and false positives limits the detection of true mutations (Xu et al. 2017). Sequencing coverage limits the detection of messages but even at high sequencing depths, it is impossible to reveal all key indices without observing many messages using the same key. Lastly, in this scheme, perturbations in cell populations can be detected by analyzing the frequency of edits; genomic bottlenecks would shift the composition of a cell population, and analyzing the editing frequencies of a key index can thus allow a sender to verify if the strain has been tampered with. On the other hand, an adversary would not be able to find, read, or modify its editing frequency. Here, Applicants implement this cryptographic scheme in living mammalian cells. Applicants demonstrate that messages can be encoded in a parallelized way through targeted deamination with Cas9 base editors (Komor et al. 2016; Gaudelli et al. 2017) and that those messages can be reliably decoded by a recipient who has access to the key. Applicants encode the same number of characters as other mammalian encoding systems (Choi et al. 2022), but instead of serial editing steps, pooled gRNAs are added in a single step, and editing is performed in parallel. Through simulation and experimental validation, Applicants further show that breaking the key is infeasible before a threshold number of messages has been observed and that the costs are high even under ideal adversary conditions and increase non-linearly at low editing frequencies. Finally, Applicants demonstrate that such a scheme can be used to detect whether a cell strain has been subsequently modified by analyzing editing frequency shifts.

Citation or identification of any document in this application is not an admission that such a document is available as prior art to the present invention.

### SUMMARY

In one aspect, as described herein, a method of encryption comprises: (a) configuring one or more nucleic acid modifying agents to edit the plurality of genomic loci according to one or more encryption keys, wherein the one or more encryption keys link encoded information to a genomic loci thereby creating a set of genomic loci coordinates that hold the encoded information and defining an allele status for each genomic loci in the set of genomic loci coordinates; and (b) editing the plurality of genomic loci by introducing the one or more nucleic acid modifying agents to a cell or population of cells, whereby information is encrypted within the one or more genomes of the cell or population of cells.

In example embodiments, the method further comprises decoding the information by observing an allele frequency at the genomic loci defined by the one or more encryption keys. In example embodiments, the method further comprises decoding the information by observing the allele status with allele detection methods. In example embodiments, the detection method is a SHERLOCK, SURVEYOR, TAQMAN, or ENGEN mutation detection kit. In example embodiments, observing the allele frequency comprises amplifying the plurality of genomic loci defined by the one or more encryption keys and sequencing the amplified genomic loci to determine the allele frequency.

In example embodiments, the encoded information comprises digital or biological data. In example embodiments, the encoded information further includes an authentication code defining an expected allele frequency, and the decoding step further comprises comparing the expected allele frequency to an observed allele frequency, wherein an increase in the observed allele frequency relative to the expected allele frequency indicates inauthentic or invalid information.

In example embodiments, the encoded information is binary encoded. In example embodiments, an edited genomic locus corresponds to a first binary value and a non-edited genomic loci corresponds to a second binary value. In example embodiments, the allelic frequency of the alleles to the one or more genomes is less than 10%, less than 5%, less than 3%, less than 2%, less than 1%, less than 0.5%, or less than 0.1%. In example embodiments, the one or more nucleic acid modifying agents are configured to edit the plurality of genomic loci according to one or more chaff edits. In example embodiments, the one or more chaff edits are interspersed among the genomic loci according to the one or more encryption keys. In example embodiments, the one or more chaff edits are randomly assigned.

In example embodiments, the encoded information is encrypted in a set of key genomic loci coordinates, the key genomic loci coordinates being a subset of the genomic loci coordinates. In example embodiments, an order of the genomic loci is randomized. In example embodiments, the edits are encoded in a set of guide RNAs and multiple edits may be optionally carried out in parallel.

In example embodiments, the edit comprises changing a single nucleobase to another nucleobase. In example embodiments, the nucleic acid modifying agent is a base editing system or a prime editing system. In example embodiments, the base editing system comprises a cytidine deaminase or an adenosine deaminase. In example embodiments, the base editing system is engineered to have a relaxed PAM requirement, multiple base editing systems having different PAM requirements are used, the base editing system is used with another nucleic acid modifying agent that has no PAM requirement or a different PAM requirement, or a combination thereof. In example embodiments, wherein the nucleic acid modifying agent is a CRISPR-Cas, Zn Finger nuclease, a TALEN, or an Omega System that directs insertion of the edit via homology directed repair and a donor template comprising one or more edits. In example embodiments, the nucleic acid modifying agent is a CRISPR-associated transposase (CAST) system that directs insertion of the edit via transposase-mediated insertion of a donor template comprising one or more edits.

In example embodiments, the one or more genomes are from a prokaryote, an eukaryote, or a combination thereof. One aspect comprises an engineered, non-naturally occurring cell, or progeny thereof, wherein the genome of the cell is modified to store encoded information encrypted according to any of the herein-mentioned methods.

In one aspect, a method of encoding an authentication signature into a biological material comprises encoding an encrypted verification signature in one or more genomes of the biological material by introducing edits using one or nucleic acid modifying agents at a plurality of genomic loci defined according to one or more encryption keys, whereby measuring the plurality of the genomic loci as defined by the one or more encryption keys can be used to identify and/or authenticate the origin or source of the biological material.

In one aspect, a method of authenticating a biological material comprises adding one or more cells to the biological material, the one or more cells comprising information encrypted in a genome(s) of the one or more cells, wherein the encrypted information is used to authenticate the biological material. In example embodiments, the one or more cells are the engineered cells described herein.

In one aspect, a method of authenticating a biological material comprises: measuring a set of genomic loci from one or more cells obtained from the biological material and as defined by one or more encryption keys, wherein at least a portion of the cells of the biological material comprises genomes previously edited with one or more nucleic acid modifying agents to encode an authentication code according to the one or more encryption keys; wherein an observed allele status at the genomic loci, in combination with the one or more encryption keys, are used to decode the authentication signature that confirms an identity of and/or authenticates an origin of the biological material.

In example embodiments, the method further comprises decoding the information by observing an allele frequency at the genomic loci defined by the one or more encryption keys. In example embodiments, observing the allele frequency comprises amplifying the plurality of genomic loci defined by the one or more encryption keys and sequencing the amplified genomic loci to determine the allele frequency. In example embodiments, measuring comprises performing an allele detection method. In example embodiments, the detection method is a SHERLOCK, SURVEYOR, TAQMAN, or ENGEN mutation detection kit.

In example embodiments, the biological material is a modified organism or a modified cell. In example embodiments, the modified organism is a modified plant. In example embodiments, the modified cell is a therapeutic cell. In example embodiments, the authentication signature further includes an authentication code defining an expected allele frequency, and wherein the decoding step further comprises comparing the expected allele frequency to an observed allele frequency, wherein an increase in the observed allele frequency relative to the expected allele frequency indicates inauthentic or invalid information. In example embodiments, the authentication signature is binary encoded.

In example embodiments, an edited genomic locus corresponds to a first binary value and a non-edited genomic loci corresponds to a second binary value. In example embodiments, the allele frequency of the edits to the one or more genomes is less than 10%, less than 5%, less than 3%, less than 2%, less than 1%, less than 0.5%, or less than 0.1%. In example embodiments, the one or more nucleic acid modifying agents are configured to edit the plurality of genomic loci according to one or more chaff edits. In example embodiments, the one or more chaff edits are interspersed among the genomic loci according to the one or more encryption keys. In example embodiments, the one or more chaff edits are randomly assigned. In example embodiments, an order of the genomic loci is randomized.

In example embodiments, the nucleic acid modifying agent is a Zn Finger nuclease, a TALEN, a meganuclease, a CRISPR-Cas system, a CAST system, ARCUS, a base editing system, a prime editing system, or a combination thereof. In example embodiments, the nucleic acid modifying agent is a base editing system or a prime editing system. In example embodiments, the base editing system comprises a cytidine deaminase or an adenosine deaminase.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-F shows a strategy for highly parallelized information encryption into the genome of cell populations. FIG. 1A. shows an overview of the encoding scheme. Each position is assigned to one genomic location key index and corresponding mutations are encoded in DNA. FIG. 1B shows implementation of information encoding. Each key site corresponds to one bit and flipped bits are genomically encoded using base editors and pools of gRNAs. FIG. 1C shows an overview of the decoding scheme of a recipient. A recipient amplifies key index areas and retrieves the message by analyzing the editing rate. FIG. 1D shows classification of editing outcomes with a cut-off of 0.1% editing. Sites were transfected in two pools of 55 gRNAs each. Editing above and below 0.1% is colored in dark grey (1) and medium grey (0), respectively, and false negatives (FNs) and false positives (FPs) are colored in light grey (FN/FP). FIG. 1E shows a ROC curve of classification of editing outcomes. FIG. 1F shows replicate correlation of editing rates between sites.

FIG. 2A-2G show secure information transfer through asymmetric difficulty of detecting genomic mutations. FIG. 2A shows an adversary aiming to break the key would be required to perform whole genome sequencing (WGS) and use variant calling software to detect installed edits. Possible outcomes are true positives (TPs), false negatives (FNs), false positives (FPs) and true negatives (TNs). FIG. 2B shows functions for the cost of an adversary without the key. FIG. 2C shows a distinction between false positives and true positives over multiple messages, with TPs shown in dark grey and FPs shown in light grey. FIG. 2D shows a minimum number of messages required to break the key for an adversary when not limited by sequencing coverage. FIG. 2E shows the sequencing cost of an adversary over different editing frequencies and coverage levels (log 10). The coverage level required for breaking the code within 30 messages for each editing frequency is boxed in black. FIG. 2F shows the difference in cost in detecting the message without the key versus with the key over various allele frequencies. FIG. 2G shows a fraction of detected mutations from whole exome sequencing data at ˜1000× coverage with variant caller Mutect and VarScan2, at ˜1000× sequencing coverage.

FIG. 3A-3F show message authentication through encrypted population allelic frequencies. FIG. 3A shows an overview of message authentication and anti-modification strategy. Cells carrying a mutation at the anti-modification strategy (AMS) site are spiked into a cell strain at a desired ratio, resulting in a defined editing frequency at the AMS site. If maintained under regular conditions, editing frequency at the AMS site is maintained. In contrast, when cells are subjected to a bottleneck, the editing frequency is perturbed. FIG. 3B shows an absolute log fold change (LFC) of editing frequency under regular passaging conditions. FIG. 3C shows an absolute log fold change (LFC) of editing when bottlenecked to 50, 100, 500 or 1000 cells. FIG. 3D shows a fraction of sites with editing changes above different log fold change (LFC) thresholds for cells after 4 passages, and cells that were to 50 and 500 cells, respectively. FIG. 3E shows at the top table the decoding of messages, showing the number of true positives (TP), false negatives (FN) and false positives (FP). FIG. 3E at the lower table shows the original message, with errors occurring during encoding or decoding shown above the cross-outs. The double errors represent the shift character for shifting to numeric values. FIG. 3F shows cell strains mixed with strain that carries edit at anti-modification site. ‘Defined edit %’ is the editing percentage as encoded in the message, ‘actual edit %’ is the experimentally observed percentage.

FIG. 4A-4B shows the results of pooled gRNA cloning and screening. FIG. 4A shows out of 381 gRNAs that were cloned, efficient PCR amplification of 318 of the corresponding genomic sites was achieved. When transfected in batches of 48 gRNAs, 224 sites showed editing greater than 0.1%, and 137 sites showed editing greater than 0.5%. Applicants further analyzed the background rates of the 138 sites with >0.5 editing and selected 111 sites with low background rates. FIG. 4B shows the editing distribution of 226 sites that showed >0.1% editing when transfected in batches of 48 gRNAs, before filtering out sites with high background.

FIG. 5 shows editing rates at different gRNA batch sizes. Editing rates were compared using the same gRNAs for different batch sizes of gRNA with the total concentration of gRNAs per transfection kept constant. Editing efficiencies were normalized to one, and the log-fold change of the editing rate was calculated.

FIG. 6A-6B show false negative rates at varying allele percentages and coverages obtained from calling variants on read files with artificially introduced mutations. FIG. 6A shows false negative rates obtained for Mutect2. FIG. 6B shows false negative rates obtained for VarScan2.

FIG. 7A-7B show a proportion of times a genomic index that is a true positive versus a false positive is called a variant. FIG. 7A shows binomial distributions of the number of times key indices would be called variants over 200 messages at false negative rates (FNR) of 50%, 75% and 85%. FIG. 7B shows binomial distributions of true positives and false positives and the proportion of times they are called variants over 10, 20 and 100 messages.

FIG. 8 shows the number of messages needed to reveal key indices using Varscan2 versus Mutect2. Values were calculated using a statistical analysis of how many messages are needed to meet an acceptable false positive rate of 100/(3*10\hat{ }9) representing roughly the number of bits in a message over the size of the human genome and a false negative threshold of 0.1 meaning 90% of key indices have been discovered.

FIG. 9 shows attacker's cost when the optimal combination of sequence coverage and number of messages is chosen. Cost with unlimited messages is shown in black boxes; cost with messages limited to max. 30 messages is shown in grey boxes.

FIG. 10 shows the cost for an adversary to break a key when messages are encrypted at various allele frequencies. Cost for VarScan2 is shown as top line and cost for Mutect2 is shown as lower line; at less than ˜2% allele frequency the cost approaches infinity when using Mutect2.

FIG. 11 shows false positive rates generated from whole exome sequencing. Whole exome sequencing was performed at 1000× coverage and variants were called using Mutect and VarScan at 1% and 0.5% allele frequency thresholds.

FIG. 12 shows a message authentication scheme. One of the key sites is specified as a message authentication site. A strain carrying a mutation at only this site is created, the editing frequency determined by sequencing, and the strain is then diluted into cell strains with encoded messages at a ratio achieving final editing frequencies as specified in the messages, e.g. in ‘HELLO WORLD!#3’ the digit 3 specifies the editing frequency. Due to genetic drift, the editing frequency is expected to be perturbed when cells are subjected to a bottleneck compared to regular growth conditions, and the editing frequency can therefore inform about the integrity of the strain.

FIG. 13 shows a modified version of the five-bit International Telegraph Alphabet no. 2 (ITA2) for converting text to binary. ‘Shift character’ enables changing between state 1 and state 2.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosure. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Genomic Encryption

Cryptography, methods and techniques for securing communication from adversarial behavior, generally relies upon the concept of computational hardness for designing encryption schemes. Computational hardness refers to the concept that an adversary is computationally limited to decrypt encrypted information. Therefore, a method of encryption, e.g., method of encoding or converting information from one form to another, must increase in computational hardness as the power of computational systems/devices increase. Consequently, embodiments disclosed herein provide genomic encryption methods and systems, which overcome computational limitation by taking advantage of the asymmetric cost of detecting variations (e.g., mutations, edits, alleles) from one genome to another.

The method of encryption generally comprises generating an encryption key, configuring one or more nucleic acid modifying agents to edit genomic loci according to the encryption key, and editing the plurality of genomic loci to encrypt information within one or more genomes of a cell or population of cells. The method may further comprise amplifying at least those genomic loci comprising the encrypted information and decoding the encrypted information by observing a detected allele frequency at the amplified genomic loci relative to the reference genome. An allele frequency refers to the number of times (e.g., percentage) a particular variant/allele at a particular genomic locus is observed over one or more genomes relative to a reference genome. The reference genome may be an unmodified genome corresponding to the genome comprising encrypted information or the reference genome is a first modified genome that is then further modified by the methods and systems described herein.

### Generation of Encryption Key

In example embodiments, an encryption key is generated. In general, an encryption key comprises a random string of bits generated to scramble and unscramble data. Typically, encryption keys are designed to be unpredictable and unique. In example embodiments, the random string of bits corresponds to the genomic loci corresponding to the encoded information. In example embodiments, the random string of bits comprise of a string of randomly selected genomic loci in sequential order (e.g., 3, 149, 864, 5090). In example embodiments, the random string of bits comprises a string of randomly selected genomic loci in non-sequential order (e.g., 149, 5090, 3, 864). The encryption key may be any size. The size may vary based on the size of the information and the encoding scheme.

In example embodiments, after a genome is sequenced, the encryption key identifies the genomic loci encoding the information. The nucleoside (e.g., allele) of the genomic loci is recorded and the message can then be decoded.

Generating the encryption key mat comprise selecting the specific genomic loci for the encoded information.

### Encryption Schemes

In general, encoded information can be encrypted with symmetric-key encryption or asymmetric-key encryption. Symmetric-key encryption comprises using the same encryption key for both encryption and decryption. A symmetric-key may be identical or undergo a transformation such that the transformed key is scrambled compared to the untransformed key (e.g. reciprocal or non-reciprocal scrambling). In example embodiments, the generated encryption key is a symmetric-key encryption.

Example symmetric-key encryption schemes include block ciphers and stream ciphers. A block cipher encryption scheme encrypts encoded information in fixed sizes (i.e., blocks). An example of block cipher encryption is Advanced Encryption Standard (AES), which typically uses a block of 128-bits. Each block can have the same encryption or each block can be encrypted independently. A stream cipher encryption scheme encrypts the encoded information one bit at a time. In general, encoded information is encrypted with a randomly generated key the same length as the encoded information. Practically, this involves the use of a “seed-key” fed to a pseudo-number generator to produce the randomly generated key that encrypts the encoded information. Decryption then involves knowing both the “seed-key” and pseudo-number generator. An example of stream cipher encryption is Rivest Cipher 4.

Asymmetric-key encryption (i.e., public key encryption) uses a pair of keys for each party associated with the encrypting or decrypting the encoded information. One of the key pairs is a public key, which is accessible by anyone and typically used for encrypting the encoded information. The other key is a private key, which is only held by the party capable of decrypting the encoded information.

Example asymmetric-key encryption schemes include integer-based cryptography and elliptic-curve encryption schemes. In general, an integer-based encryption scheme uses “hard” mathematical problems to encrypt encoded information. Example “hard” problems include factoring and discrete logarithm problems. For example, in the case of factoring, a public key may comprise the product of two private keys which comprise large prime numbers. To discover the private key, the public key must be factored, and the difficulty of factoring grows exponentially with length. An example integer-based encryption scheme includes the RSA algorithm. An elliptic-curve encryption scheme encrypts encoded information via a randomly produced private key and a public key corresponding to integer coordinates on an elliptic-curve (e.g., y²=x³+ax+b) dotted against itself the randomly generated private key number of times. Different curves affect the strength of the encryption, speed it can be decrypted, and different key lengths. Example elliptic-curve encryption schemes include Elliptic Curve Digital Signature Algorithm, Elliptic Curve Integrated Encryption Scheme, and Elliptic-curve Diffie-Hellman algorithm.

Asymmetric-key encryption can be further secured by using authentication key pairs. In general, authentication key pairs rely on two pairs of public and private keys. In example embodiment, the public key in one of the authentication key pairs only encrypts while the corresponding private key only decrypts. The other public key in the other authentication key pair only decrypts while the corresponding private key only encrypts. Thus, encoded information is encrypted with a public key and an authentication message is encrypted with a private key. The receiving party can then first verify the authenticity of the message with a public key that decrypts the authentication message and then decrypts the encoded information with the private key.

### Chaffing and Winnowing

In example embodiments, the methods and systems described herein comprise of chaffing and winnowing (CW). CW arises from the observation that harvested grain (i.e. encoded information) remains mixed with inedible chaff (i.e., information intended to obfuscate) wherein the grain is difficult to distinguish from the chaff. The valuable grain is separated from the chaff by a process called winnowing. Similarly, in the context of cryptography, encoded information is interspaced with information intended to obfuscate (i.e., conceal) the encoded information from adversarial parties. The party who receives the encrypted message simply needs to “winnow” (i.e., remove) the “chaff” (i.e., obfuscating information) to retrieve the encoded information according to a key. In example embodiments, CW can be considered as a type of symmetric encryption. See e.g., Rivest, Ronald L. “Chaffing and winnowing: Confidentiality without encryption.” CryptoBytes (RSA laboratories) 4.1 (1998): 12-17 and Bellare, Mihir, and Alexandra Boldyreva “The security of chaffing and winnowing.” International Conference on the Theory and Application of Cryptology and Information Security. Springer, Berlin, Heidelberg, 2000.

In example embodiments, the one or more nucleic acid modifying agents are configured to edit the plurality of genomic loci according to one or more chaff edits. A chaff edit comprises any edit that does not correspond to the encoded information and is intended to obfuscate and/or conceal the encoded information. In the context of genomic cryptography, information is encoded and an encryption key links the encoded information to genomic loci, which has been modified according to the encoded information. The one or more chaff edits are further incorporated into the genome corresponding to loci that is not according to the encryption key. The chaff edits are interspersed among the genomic loci according to the encryption key, either randomly or based on some pattern. The one or more chaff edits are intended to be removed/ignored upon decryption of the encoded information. For example, chaff edits are not sequenced.

In example embodiments, the encryption key comprises one or more chaff edits. In example embodiments, wherein the encryption key comprises one or more chaff edits, the encryption key identifies these edits as separate from the encoded information and may need to be removed/ignored. In example embodiments, the encryption key does not comprise chaff edits. In example embodiments, the one or more chaff edits are randomly assigned to one or more genomic loci. In example embodiments, the one or more chaff edits are assigned to one or more genomic loci based on a pattern.

### Genomic Loci Coordinates

In example embodiments, generating an encryption key, the encryption key linking encoded information to a genomic locus thereby creating a set of genomic loci coordinates that hold the encoded information and defining an allele status for each genomic loci in the set of genomic loci coordinates. A genomic locus or genomic loci refers to one (i.e., locus) or more (i.e., loci) specific and fixed positions of a gene on a chromosome. A genomic locus may be labeled using any suitable nomenclature. For example, a genomic locus may be identified (e.g., indexed, labeled) by the chromosome number/identifier (e.g., 7, chromosome 7), the arm (e.g., p-arm for the short arm, q-arm for the long arm), region (e.g., region 3), band (e.g., band 3), sub-band (e.g., sub-band 4), sequential location number (e.g., 25,431,736; 46,767,848; 125,005,423), or any combination thereof. An allele status is a designation of the variant at a particular genomic locus. For example, the allele status at genomic locus X may be A, G, T, C, and/or purine or pyrimidine.

In example embodiments, genomic encryption comprises encoding information to genomic loci. Encoding information comprises changing (e.g., altering, transforming, manipulating) the format of information from one form to another for optimal transmission and/or storage. In example embodiments, encoding information comprises character encoding, which comprises assigning numbers to characters (e.g., letters, numbers, punctuation, and/or symbols). Character encoding comprises selecting a code unit (e.g., code value or “word size”) of the character encoding scheme (e.g., 5-bit, 7-bit, 8-bit, 16-bit, 32-bit, 64 bit, etc. binary encoding) and transforming character set (i.e., information to be encoded) into coded character set (i.e., the set of unique numbers corresponding to the character set). In example embodiments, one or more characters are encoded using multiple code units, which results in a variable-length encoding scheme. Methods of encoding information are well known in the art (e.g., Unicode, ASCII, International Telegraph Alphabet no. 2 (ITA2)) and will not be described further herein.

In an example embodiment, information encoded to a genomic locus comprises a binary encoding scheme where an allele (e.g., the variant/mutation at a particular genomic locus) corresponds to a 0 or 1. In an example embodiment, wherein a genome locus allele corresponds to a binary scheme, multiple genomic loci correspond to a bit scheme. For example, an 8-bit binary scheme would require 8 genomic loci per character.

In an example embodiment, the encoding scheme is a 4-base number system. In a 4 base system a genomic locus (e.g., A, T, C, G) corresponds to 0, 1, 2, 3 or any variation of four numbers. For example, in a 4-base 4-bit encoding scheme 256 characters can be encoded, while in a binary 4-bit encoding scheme only 16 characters can be encoded.

These examples are non-limiting and additional encoding schemes would be readily understood by one skilled in the art.

### Type of Information

The encoded information can comprise any type of information. For example, encoded information may be qualitative information (i.e., categorical data), quantitative information, or a combination thereof. Qualitative information may comprise information that cannot be counted or measured easily using numbers and is typically divided by category (e.g., the color of objects). Qualitative information can be classified as nominal information or ordinal information. Nominal information may comprise qualitative information that does not have a natural or innate ordering (e.g., ranking colors). Ordinal information may comprise qualitative information that does have a natural ordering (e.g., a grading system). Quantitative information may comprise information that is naturally organized by numerical values. Quantitative information can be classified as discrete information or continuous information. Discrete information may comprise information corresponding to integer or whole numerical values. Continuous information may comprise information corresponding to fractional numbers.

In example embodiments, the encoded information comprises digital or biological data. Digital data comprises a string of discrete characters and may comprise any type of information. Digital data may be compressible such that the encoded information uses fewer bits than the original message. Digital data may comprise the types of information described herein. Biological data may comprise information derived from organisms. Information derived from organisms may include but is not limited to atomic structure (e.g., types of atoms), molecular structure (e.g., type of molecules), sequence (e.g., nucleic or amino), genome data, three-dimensional structure (e.g., secondary, tertiary structure), location of components, products (e.g., medicinal compound), or any combination thereof. In example embodiments, the encoded information comprises biological information about the organism from which the modified genome is derived. In example embodiments, the digital data may comprise biological data.

In example embodiments, the encoded information may comprise one or more messages, barcodes, or combination thereof. A message may comprise any information (e.g. any combination of characters) shared between two or more parties. In an example embodiment, the encoded information comprises a message and a barcode. The barcode comprises an authentication code (any 1 or more characters) that verifies the message.

In example embodiments, the information encoded onto the genomic loci comprises information about the genome it is encoded onto. For example, the encoded information may comprise a message about the genome with the encoded information. In an example embodiment, the encoded information may comprise a barcode corresponding to the genome encoded with the barcode. In an example embodiment, the encoded information may comprise a message and barcode about the genome with the encoded information.

Encoding Information in gDNA Using Nucleic Acid Modifying Agents

In an example embodiment, editing the plurality of genomic loci by introducing the one or more nucleic acid modifying agents to a cell or population of cells. The nucleic acid modifying agent is programmed to edit the genomic loci according to the generated encryption key described above. Edits may be mutations or deletions of one or more bases, conversion of one or more nucleobases to another one or more nucleobases, and/or an insertion of one or more bases or a polynucleotide sequence at the genomic loci. The nucleic acid modifying agent may comprise a programmable nuclease which can be configured to encode the encrypted information at the specified genomic loci. Example programmable nucleases include CRISPR-Cas, Omega systems, Zn Finger Nucleases, TALENs, and meganucleases. The information may be encoded by non-homologous end joining (NHEJ) or homology directed repair (HDR) using the programmable nucleases single strand or double-strand DNA nuclease activity. Alternatively, the programmable nuclease may be rendered fully or partially catalytically inactive and paired with another functional domain that encodes the information at the genomic loci. Functional domains that may be used for this purpose include, but are not limited to, nucleobase deaminases, reverse transposases, polymerases, ligases, topoisomerases, and retrotransposons. In one example embodiment, the nucleic acid modifying agent is a base editor. In another example embodiment, the nucleic acid modifying agent is a prime editor.

### Base Editing

In one example embodiment, the nucleic acid modifying agent is a base editing system. As used herein, “base editing” refers generally to the process of polynucleotide modification via nucleotide deaminase that does not include excising nucleotides to make the modification. Base editing can convert base pairs at precise locations without generating excess undesired editing byproducts that can be made using traditional double-stranded DNA cleavage. In one example embodiment, a nucleotide deaminase is connected or fused to a programmable nuclease such as a catalytically inactive Cas, but other programmable nucleases may be used in place of Cas.

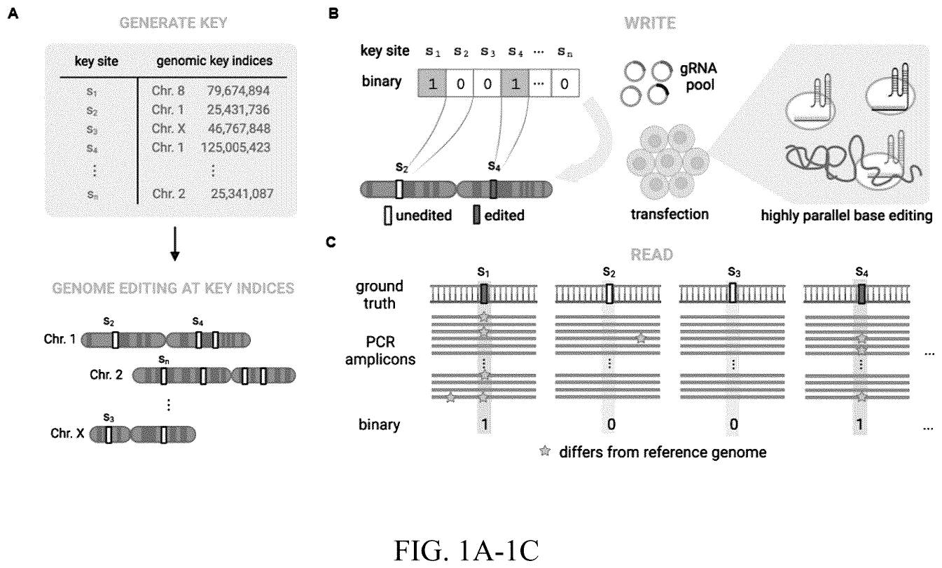

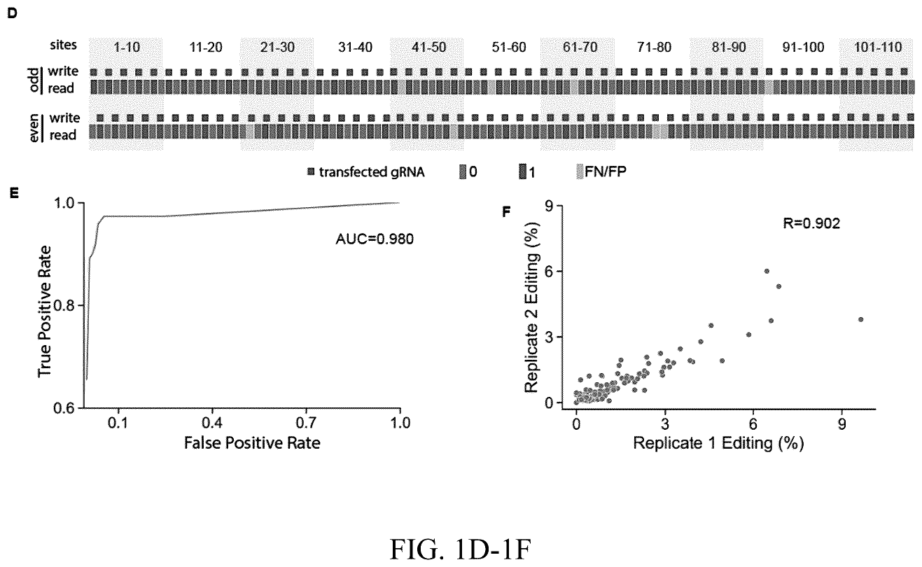

In one example embodiment, the nucleotide deaminase may be a DNA base editor used in combination with a DNA binding protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, G to A, and C-to-U, and A-to-U). Rees and Liu. 2018. Nat. Rev. Genet. 19 (12): 770-788, particularly at Figures 1b, 2a-2c, 3a-3f, and Table 1. In some embodiments, the base editing system includes a CBE and/or an ABE. In some embodiments, a polynucleotide of the present disclosure described elsewhere herein can be modified using a base editing system. Rees and Liu. 2018. Nat. Rev. Gent. 19 (12): 770-788. Base editors also generally do not need a DNA donor template and/or rely on homology-directed repair. Komor et al. 2016. Nature. 533:420-424; Nishida et al. 2016. Science. 353; and Gaudelli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas, can have nickase functionality, and can generate a nick in the non-edited DNA strand to induce cells to repair the non-edited strand using the edited strand as a template. Komor et al. 2016. Nature. 533:420-424; Nishida et al. 2016. Science. 353; and Gaudelli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In example embodiments, the base editing system further converts C to G. For example, to perform this conversion, the base editing system further comprises a uracil binding protein as described in International Patent Publication No. WO2018/165629A1, incorporated herein by reference. In example embodiments, the base editing system further converts A to T or T to A. For example, to perform this conversion, the base editing system further comprises an adenosine methyltransferase, a thymine alkyltransferase, or an oxidase as described in US Patent Application Publication No US20220170013A1, International Patent Publication No. WO2020181178A1 and WO2020181202A1, all of which are incorporated herein by reference. In example embodiments, the base editing system further converts G to T and C to A. For example, to perform this conversion, the base editing system further comprises guanine oxidase as described in US Patent Application Publication No US20220282275 A1, incorporated herein by reference. In example embodiments, the base editing system further converts A to C or T to G. For example, to perform this conversion, the base editing system further comprises adenine oxidase as described in International Patent Publication No WO 2020181180 A1, incorporated herein by reference. In an example embodiment, the base editing system further converts T to G or A to C. For example, to perform this conversion, the base editing system further comprises a transglycosylase domain as described in International Patent Publication No WO 2021030666 A1, incorporated herein by reference.

In example embodiments, the base editing system may be further modified. For example, base editing system may be further modified using phage-assisted continuous evolution (PACE) as described in US Patent Application Publication US20200172931 A1 and International Patent Publication No WO 2021158921 A3, both of which are incorporated herein by reference. The base editing system may be further modified by including a Gam protein as described in U.S. Pat. No. 1,131,953 2B2, incorporated herein by reference. The base editing system may be further modified by making mutations that increase DNA efficiently, reduce RNA off-target editing activity, reduce off-target DNA editing activity, indel byproduct formation, or any combination thereof as described in US Patent Application Publication No. US20220307003 A1, incorporated herein by reference.

General guidance on classes of base editors, relevant advances and delivery strategies is described in Porto E M, et al. Base editing: advances and therapeutic opportunities. Nat Rev Drug Discov. 2020 December; 19 (12): 839-859, incorporated herein by reference.

An example method for delivery of base-editing systems, including use of a split-intein approach to divide CBE and ABE into re-constitutable halves, is described in Levy et al. Nature Biomedical Engineering doi.org/10.1038/s41441-019-0505-5 (2019), which is incorporated herein by reference.

In example embodiments, the base editing system is engineered to have a relaxed PAM requirement, multiple base editing systems having different PAM requirements are used, the base editing system is used with another nucleic acid modifying agent that has no PAM requirement or a different PAM requirement, or a combination thereof.

PAM requirements may be altered to particularly encrypt information or encrypt particular information. Accordingly, known methods to alter PAM requirements may be used to alter, modify, or otherwise change the PAM requirement of a base editing system. See e.g., Leenay, R. T.; Beisel, C. L. Deciphering, Communicating, and Engineering the CRISPR PAM. Journal of Molecular Biology, 2017, 429, 177-191, Fischer, S.; et al., A. An Archaeal Immune System Can Detect Multiple Protospacer Adjacent Motifs (PAMs) to Target Invader DNA. Journal of Biological Chemistry, 2012, 287, 33351-33363 and Niewoehner, O.; Jinek, M. Specialized Weaponry: How a Type III-A CRISPR-Cas System Excels at Combating Phages. Cell Host &amp; Microbe, 2017, 22, 258-259, all of which are incorporated herein by reference.

In some instances, the PAM requirement may be relaxed to increase the overall targetable sequences. For example, the PAM requirement may be changed from NGG to NGN. A relaxed PAM requirement can be designed and/or optimized for a given encryption scheme. See e.g., Huang, X.; et al. Decoding CRISPR-Cas9 PAM Recognition with UniDesign, 2023, which is incorporated herein by reference.

In some instances, the base editing system is used with another nucleic acid modifying agent that has no PAM requirement or a different PAM requirement. A system with no PAM requirement may increase the enzymatic activity of the base editing system and/or increase the capability of the base editing system to use all or nearly all PAMs. See e.g., Walton, R. T.; et al. Unconstrained Genome Targeting with Near-PAMless Engineered CRISPR-Cas9 Variants. Science, 2020, 368, 290-296 and Collias, D., Beisel, C. L. CRISPR technologies and the search for the PAM-free nuclease. Nat Commun 12, 555 (2021), all of which are incorporated herein by reference.

### ARCUS Based Editing

In one example embodiment, the base editor is an ARCUS base editing system. Exemplary methods for using ARCUS can be found in U.S. Pat. No. 10,851,358, US Publication No. 2020-0239544, and WIPO Publication No. 2020/206231 which are incorporated herein by reference.

### Prime Editors

In one example embodiment, the nucleic acid modifying agent is a prime editing system. See e.g. Anzalone et al. 2019. Nature. 576:149-157 and U.S. Pat. No. 11,447,770 B1, incorporated herein by reference. In one example embodiment, a genomic sequence in a target gene or sequence controlling expression of the target gene is edited using a prime editing system. Like base editing systems, prime editing systems can be capable of targeted modification of a polynucleotide without generating double stranded breaks. Further prime editing systems are capable of all 12 possible combination swaps. Prime editing may operate via a “search-and-replace” methodology and can mediate targeted insertions, deletions, of all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present disclosure include these and variants thereof. Prime editing can have the advantage of lower off-target activity.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576:149-157, particularly at Figures 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associated with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

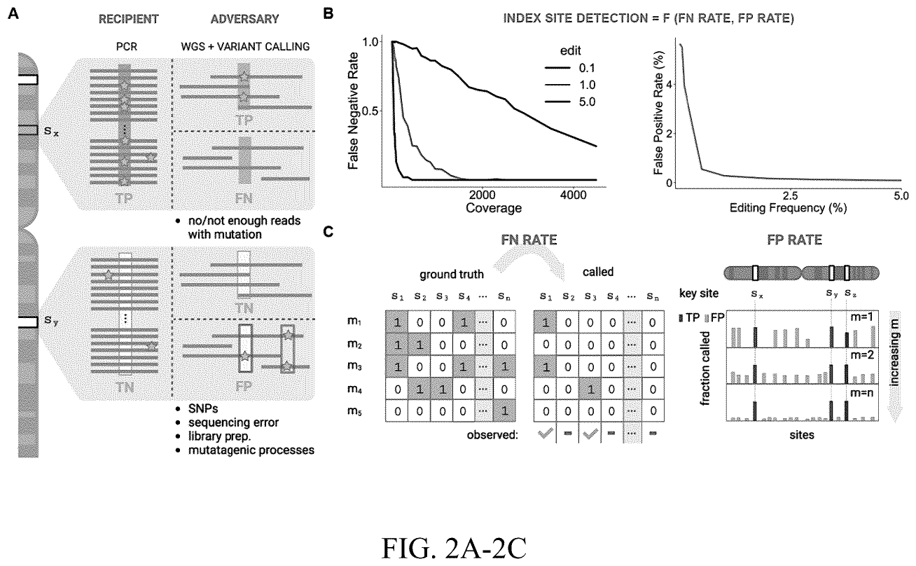

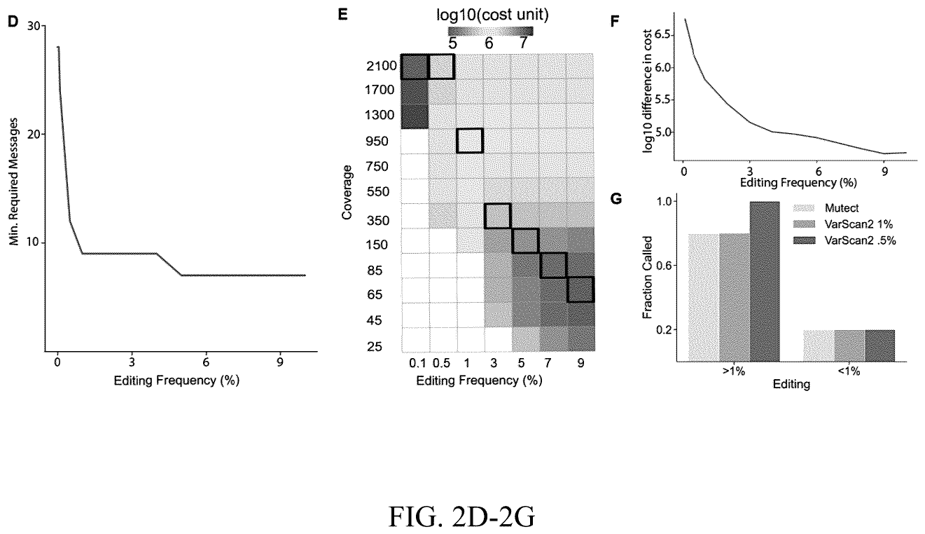

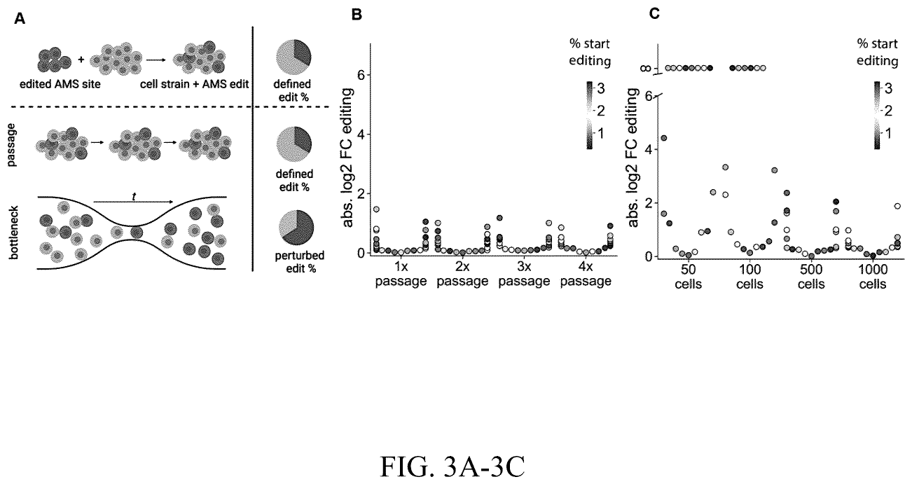

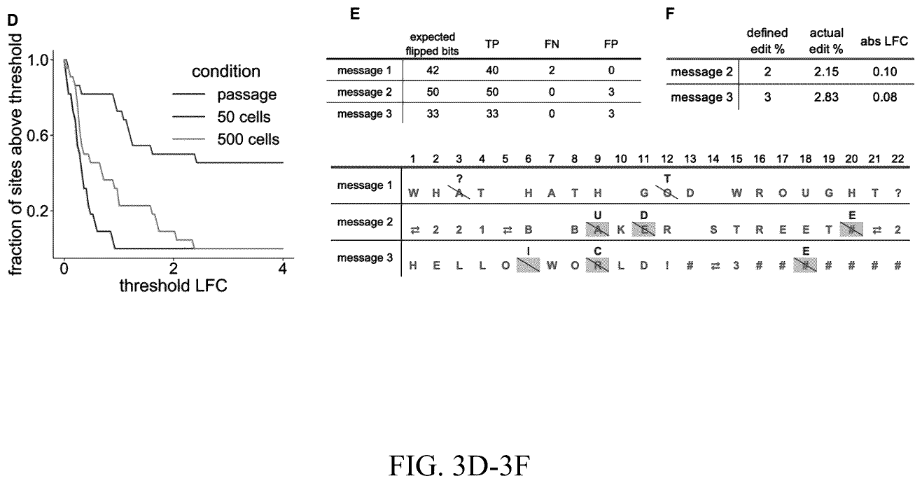

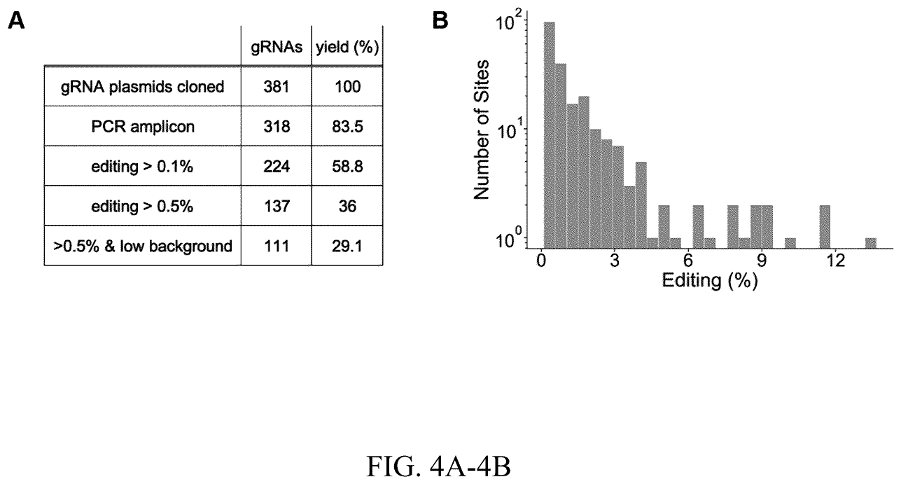

In some embodiments, the prime editing system can be a PE1 system or variant thereof, a PE2 system or variant thereof, or a PE3 (e.g., PE3, PE3b) system. See e.g., Anzalone et al. 2019. Nature. 576:149-157, particularly at pgs. 2-3, Figs. 2a, 3a-3f, 4a-4b, Extended data Figs. 3a-3b, 4,

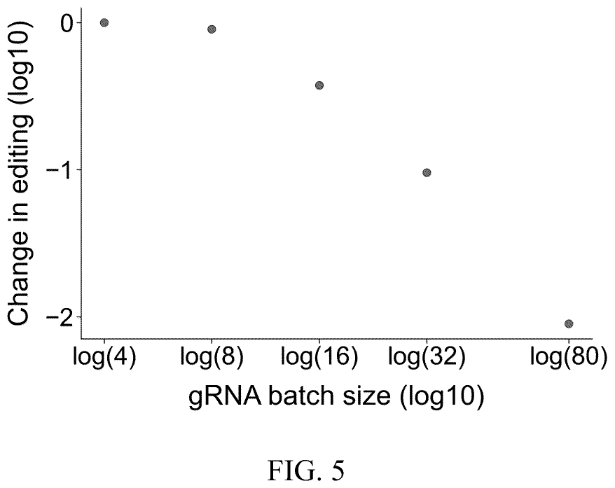

The peg guide molecule can be about 10 to about 200 or more nucleotides in length. Optimization of the peg guide molecule can be accomplished as described in Anzalone et al. 2019. Nature. 576:149-157, particularly at pg. 3, FIG. 2a-2b, and Extended Data FIGS. 5a-c.

In example embodiments, the prime editing system is capable of simultaneous editing of both strands of a target double-stranded nucleotide sequence. For example, to accomplish simultaneous editing of both strands of a target double-stranded nucleotide sequence, a prime editing system may comprise a first and second prime editor complex as described in International Patent Publication No. WO 2021226558 A8, incorporated herein by reference.

In example embodiments, the prime editing system comprise further modifications. For example, the modifications may comprise improved editing efficiency and/or reduced indel formation as described in International Patent Publication No. WO 2022150790 A3, incorporated herein by reference.

In example embodiments, the prime editing system comprises modifications to the prime editing guide RNA. For example, a modification to the prime editing guide RNA may comprise at least one nucleic acid extension arm comprising a DNA synthesis template and a primer binding site, wherein the extension arm comprises a nucleic acid moiety attached thereto selected from the group consisting of a toe-loop, hairpin, stem-loop, pseudoknot, aptamer, G-quadraplex, tRNA, riboswitch, or ribozyme as described in International Patent Application WO 2022067130 A3, incorporated herein by reference.

In example embodiments, the prime editing system comprises a catalytically active Cas polypeptide instead of a Cas nickase, see e.g., International Patent Publication No WO 2022203905 A1, incorporated herein by reference.

General guidance, current developments, improvements, variations, and delivery methods are described in Chen, P. J., Liu, D. R. Prime editing for precise and highly versatile genome manipulation. Nat Rev Genet (2022), incorporated herein by reference. For example, Chen the addition of NLS tags to PE2 prime editing systems, apegRNAs which stabilize the secondary structure of the second stem-loop within the pegRNA scaffold, PE variants with comprising chromatin modulating peptides, and adenoviral delivery of prime editing systems, for example.

### NHEJ and HDR Templated Encoding

### Cas Proteins

In example embodiments, the one or more nucleic acid modifying agents to edit the plurality of genomic loci according to the encryption key is a CRISPR-Cas system. In general, a CRISPR-Cas or CRISPR system as used herein and in documents, such as International Patent Publication No. WO 2014/093622 (PCT/US2013/074667) and U.S. Pat. No. 10,669,540 B2 incorporated herein by reference, refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e., the guide sequence is designed to have complementarity and to which the effector function mediated by the complex comprising CRISPR effector protein and a gRNA is to be directed. In some embodiments, a target sequence is located in the nucleus or cytoplasm of a cell.

The RNA-guided nucleases herein may be identified by their proximity to cas1 genes, for example, though not limited to, within the region 20 kb from the start of the cas1 gene and 20 kb from the end of the cas1 gene. In certain embodiments, the RNA-guided nuclease comprises at least one HEPN domain and at least 500 amino acids, and protein is naturally present in a prokaryotic genome within 20 kb upstream or downstream of a Cas gene or a CRISPR array. Non-limiting examples of RNA-guided nucleases include Cas1, Cas1B, Cas2, Cas3, Cas4, Cas5, Cas6, Cas7, Cas8, Cas9 (also known as Csn1 and Csx12), Cas10, Cas12 (e.g., Cas12a, Cas12b, Cas12c, Cas12d), Cas13 (e.g., (Cas13a, Cas13b, Cas13c, Cas13d), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. See e.g., U.S. Pat. No. 11,384,344 B2 incorporated herein by reference. In one example embodiment, the RNA-guided nucleases may be the nuclease in any CRISPR-Cas system. In another example embodiment, the CRISPR system may be a class 2 CRISPR-Cas system, including Type II, Type V and Type VI systems. In certain example embodiments, the RNA-guided nuclease may be a is a Cas9, a Cas12a, Cas12b, Cas12c, Cas12d, Cas13a, Cas13b, Cas13c, or Cas13d system. For example, the RNA-guided nuclease may be Cas9, a Cas12a, Cas12b, Cas12c, Cas12d, Cas12k, a CasX, a CasY, a CasF, a MAD7, a Cas13a, Cas13b, Cas13c, or Cas13d.

In certain example embodiments, the RNA-guided nuclease is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Non-Homologous End-Joining

In an embodiment, nuclease-induced non-homologous end-joining (NHEJ) can be used to edit a plurality of genomic loci. Nuclease-induced NHEJ can also be used to edit (e.g., delete/insert) an allele in a gene of interest.

In the context of genomic encryption, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the removal and addition of nucleotides, at one or both strands, prior to rejoining of the ends. This results in the presence of an edit to an allele in the DNA sequence at the site of the NHEJ repair.

NHEJ edits tend to be short and often include short duplications of the sequence immediately surrounding the break site. However, it is possible to obtain large edits, and in these cases, the edited sequence has often been traced to other regions of the genome or to plasmid DNA present in the cells.

In some examples, the systems herein may introduce one or more indels via NHEJ pathway and insert sequence from a combination template via HDR.

### Homology Directed Repair

In an embodiment, nuclease-induced homology-directed repair (HDR) can be used to edit a plurality of genomic loci. Nuclease-induced HDR can also be used to edit (e.g., delete/insert) an allele in a gene of interest.

In the context of genomic encryption, a double strand break (DSB) in DNA initiates HDR which joins together the two ends in the presence of a nucleic acid called a homologous duplex template (HDT). Upon the DSB, a 3′ overhang is created by resecting 5′-ended DNA strand at the break. The HDT pairs with one strand of the homologous DNA duplex and displaces the other strand. The DNA is then repaired according to the HDT thereby creating an edit in the plurality of genomic loci. In an example embodiment, the one or more nucleic acid modifying agents comprise a homologous recombination donor template comprising a donor polynucleotide sequence for editing a plurality of genomic loci. See e.g., Liu, M.; et al. Methodologies for Improving HDR Efficiency. Frontiers in Genetics, 2019, 9.

The activity of NHEJ and HDR DSB repair can vary by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting editing that require precise genome modifications to mitotic cells. See e.g., Ciccia, A. & Elledge, S. J. Molecular cell 40, 179-204 (2010); Chapman, J. R., et al. Molecular cell 47, 497-510 (2012).

The efficiency of correction via HDR may be controlled by the epigenetic state or sequence of the targeted locus, or the specific repair template configuration (single vs. double stranded, long vs. short homology arms) used, see e.g., Hacein-Bey-Abina, S., et al. The New England journal of medicine 346, 1185-1193 (2002) and Gaspar, H. B., et al. Lancet 364, 2181-2187 (2004); Beumer, K. J., et al. G3 (2013). The relative activity of NHEJ and HDR machineries in target cells may also affect gene editing efficiency, as these pathways may compete to resolve DSBs, see e.g., Beumer, K. J., et al. Proceedings of the National Academy of Sciences of the United States of America 105, 19821-19826 (2008). Thus, these differences can be kept in mind when designing, optimizing, and/or selecting a NHEJ and/or HDR system.

### CRISPR Associated Transposase (CAST) Systems

In one example embodiment, the nucleic acid modifying agent is a CRISPR associated transposase system (CAST). In one example embodiment, a CAST system is used to edit a plurality of genomic loci according to the encryption key. A CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. Example CAST systems are disclosed in Klompe et al. “Transposon-encoded CRISPR-Cas systems direct RNA-guided DNA integration,” Nature, 571:219-225 (2019); Saito et al. “Dual modes of CRISPR-associated transposon homing” Cell, 184 (9): 2441-2453 (2021); Cameron et al. “Harnessing Type 1 CRISPR-Cas systems for human genome engineering,” Nat Biotechno, 37:1471-1477 (2019); Halpin-Healy et al. “Structural basis of DNA targeting by transposon-encoded CRISPR-Cas systems” Nature, 577:271-274 (2020), Klompe et al. “Evolutionary and mechanistic diversity of Type I-F CRISPR-associated transposons,” Mol Cell, 82:616-628 (2022) An example Class 2 system is described herein and in Strecker et al. “RNA-guided DNA insertion with CRISPR-associated transposase,” Science, 365 (6448): 48-53 (2019)), and PCT/US2019/066835 which are incorporated herein by reference.

The systems herein may comprise one or more components of a transposon and/or one or more transposases. The transposases in the systems herein may be CRISPR-associated transposases (also used interchangeably with Cas-associated transposases, CRISPR-associated transposase proteins herein) or functional fragments thereof. CRISPR-associated transposases may include any transposases that can be directed to or recruited to a region of a target polynucleotide by sequence-specific binding of a CRISPR-Cas complex. CRISPR-associated transposases may include any transposases that associate (e.g., form a complex) with one or more components in a CRISPR-Cas system, e.g., Cas protein, guide molecule etc.). In certain example embodiments, CRISPR-associated transposases may be fused or tethered (e.g. by a linker) to one or more components in a CRISPR-Cas system, e.g., Cas protein, guide molecule etc.).

The term “transposon”, as used herein, refers to a polynucleotide (or nucleic acid segment), which may be recognized by a transposase or an integrase enzyme and which is a component of a functional nucleic acid-protein complex (e.g., a transpososome, or transposon complex) capable of transposition. Transposons employ a variety of regulatory mechanisms to maintain transposition at a low frequency and sometimes coordinate transposition with various cell processes. Some prokaryotic transposons can also mobilize functions that benefit the host or otherwise help maintain the element.

The term “transposase” as used herein refers to an enzyme, which is a component of a functional nucleic acid-protein complex capable of transposition and which mediates transposition. The transposase may comprise a single protein or comprise multiple protein subunits. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form a transpososome complex and to perform a transposition reaction.

In some embodiments, the system comprises one or more Tn7 transposase polypeptides. In some embodiments, three transposon-encoded proteins form the core transposition machinery of Tn7: a heteromeric transposase (TnsA and TnsB) and a regulator protein (TnsC). In addition to the core TnsABC transposition proteins, Tn7 elements encode dedicated target site-selection proteins, TnsD and TnsE. In conjunction with TnsABC, the sequence-specific DNA-binding protein TnsD directs transposition into a conserved site referred to as the “Tn7 attachment site,” attTn7 via its C-terminal that binds directly with DNA. TnsD (e.g. TnsD1 and TnsD2) is a member of a large family of proteins that also includes TniQ (e.g. TniQ1 and TniQ2), a protein found in other types of bacterial transposons. TniQ has been shown to target transposition into resolution sites of plasmids. TniQ works with Cascade/Cas12k (CAST) for RNA guided transposition. TniQ is a shorter version of TnsD comprising around 300 amino acids. TniQ also comprises a N-terminal similar to that of TnsD but lacks the corresponding C-terminal. Therefore, TniQ interacts with the Cascade to bind DNA. The addition of a TnsD C-terminal to a TniQ would amount to a TnsD. As used herein, a TniQ transposase may be a TnsD transposase. In some examples, the Tn7 comprises a transposase that has the activities of typical TnsA and TnsB. In an example embodiment, the transposase that has the activities of typical TnsA and TnsB is a fusion protein and may also be referred to as TnsAB. In some examples, the transposase is not a fusion protein of typical TnsA and TnsB. An example of the transposase is TnsA in IB20. Examples of Tn7 transposase polypeptides include but are not limited to TnsA, TnsB, TnsC, TniQ, TnsD, and TnsE.

As used herein, a right end sequence element or a left end sequence element are made in reference to an example Tn7 transposon. The general structure of the left end (LE) and right end (RE) sequence elements of canonical Tn7 is established. Tn7 ends comprise a series of 22-bp TnsB-binding sites. Flanking the most distal TnsB-binding sites is an 8-bp terminal sequence ending with 5′-TGT-3′/3′-ACA-5′. The right end of Tn7 contains four overlapping TnsB-binding sites in the ˜90-bp right end element. The left end contains three TnsB-binding sites dispersed in the ˜150-bp left end of the element. The number and distribution of TnsB-binding sites can vary among Tn7-like elements. End sequences of Tn7-related elements can be determined by identifying the directly repeated 5-bp target site duplication, the terminal 8-bp sequence, and 22-bp TnsB-binding sites (Peters J E et al., 2017). Example Tn7 elements, including right end sequence element and left end sequence element include those described in Parks A R, Plasmid, 2009 January; 61 (1): 1-14.

As used herein, Tn7 transposons and transposases include Tn7-like transposons and transposases. For further guidance on CAST nucleic acid modifying agent see U.S. Pat. No. 11,384,344 B2, incorporated herein by reference.

### Other Programmable Nucleases

The CRISPR-Cas based embodiment discussed above may also be carried out with alternative programmable nucleases that mediate NHEj, HDR, base editing, or donor polynucleotide insertion and as further discussed below.

### Zinc Finger Nucleases

In some embodiments, the nucleic acid modifying agent is a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, the nucleic acid modifying agent is a TALE nuclease or TALE nuclease system. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the disclosure will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the disclosure may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the disclosure.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

In some embodiments described herein, the TALE polypeptides of the disclosure include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the disclosure may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4× domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the disclosure may include any combination of the activities described herein.

### Meganucleases

In some embodiments, the nucleic acid modifying agent is a meganuclease or system thereof. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### Omega Systems

In example embodiments, the one or more nucleic acid modifying agents to edit the plurality of genomic loci according to the encryption key is an Omega system. In general, an Omega system (i.e., obligate mobile element-guided activity) is a class of transposon-encoded RNA-guided nucleases. Non-limiting examples of an Omega system include IscB, IsrB, and TnpB.

In example embodiments, the Omega system comprises an IscB. Unless indicated otherwise, the term “IscB polypeptide” will be intended to include IscB or IsrB. In one embodiment, IscB polypeptides of the present disclosure may comprise a split RuvC nuclease domain comprising RuvC-1, Ruv-C II, and Ruv-C III subdomains. Some IscB proteins may further comprise a HNH endonuclease domain. In one example embodiment, the RuvC endonuclease domain is split by the insertion of a bridge helix, a HNH domain, or both. However, unlike Cas9, IscB polypeptides do not contain a Rec domain. In addition, IscB polypeptides may further comprise a conserved N-terminal domain (also referred to herein as a PLMP domain), which is not present in Cas9 proteins. IscB proteins may also further comprise a conserved C-terminal domain.

The Cas IscB nucleic acid-guided nuclease may comprise one or more domains, e.g., one or more of a X domain (e.g., at N-terminus), a RuvC domain, a Bridge Helix domain, and a Y domain (e.g., at C-terminus). In one example embodiment, an IscB polypeptide comprises, moving from the N- to C-terminus, a PLMP domain, a RuvC-I subdomain, a bridge helix, a RuvC-II subdomain, a HNH domain, a RuvC-III subdomain, and a C terminal domain.

In example embodiments, the Omega system comprises an IsrB. As noted above, IsrBs are homologs of IscB polypeptides. IsrB polypeptides comprise the PLMP and RuvC domains but do not comprise a HNH domain. In one embodiment, the IsrB polypeptide comprises a PLMP domain and a split RuvC but lacks the HNH domain present between the RuvC-II and III subdomains in IscB polypeptides. In one embodiment, the IsrB is an ωRNA guided nickase. In one embodiment, the ωRNA guided IsrB nicks a DNA target. In one embodiment, the DNA target is a dsDNA and the nicks occur on the non-target strand of the dsDNA target. In one embodiment, the IsrB nicks the dsDNA in a guide and TAM specific manner. Accordingly, applications where a nickase is utilized can be used with the IsrB polypeptides detailed herein in a manner functionally similar to an IscB that has been inactivated at the HNH domain.

TnpB polypeptides of the present disclosure may comprise a Ruv-C-like domain. The RuvC domain may be a split RuvC domain comprising RuvC-I, RuvC-II, and RuvC-III subdomains. The TnpB may further comprise one or more of a HTH domain, a bridge helix domain and a zinc finger domain. TnpB polypeptides do not comprise an HNH domain. In one example embodiment, TnpB proteins comprise, starting at the N-terminus a HTH domain, a RuvC-I sub-domain, a bridge helix domain, a RuvC-II sub-domain, a zinger finger domain, and a RuvC-III sub-domain. In one example embodiment, the RuvC-III sub-domain forms the C-terminus of the TnpB polypeptide.

The Omega systems herein may further comprise one or more nucleic acid components, which are also referred to herein as omega RNA (ωRNA). Such nucleic acid components may comprise RNA, DNA, or combinations thereof and include modified and non-canonical nucleotides as described further below. The ωRNA can comprise a reprogrammable spacer sequence and a scaffold that interacts with the Omega system. ωRNA may form a complex (Ω complex) with an Omega polypeptide, and direct sequence-specific binding of the complex to a target sequence of a target polynucleotide. In one example embodiment, the ωRNA is a single molecule comprising a scaffold sequence and a spacer sequence. In certain example embodiments, the spacer is 5′ of the scaffold sequence. In one example embodiment, the ωRNA may further comprise a conserved nucleic acid sequence between the scaffold and spacer portions. The secondary structure of ωRNAs comprise multi-stem regions and pseudoknots. Omega systems cleave a target in an ωRNA-dependent manner upstream of the target-adjacent motif (TAM). An Omega system can use multiple trans-encoded ωRNA to cleave multiple targets.

For general guidance see e.g., Altae-Tran et al., Science 374, 57-65 (2021), International Patent Publication No WO 2022/087494 A1, WO 2022/159892 A1, and WO 2023/114872 A2, all of which are incorporated herein by reference.

### Parallel Encoding

In example embodiments, the edits are encoded in a set of guide RNAs (gRNAs) and multiple edits may be optionally carried out in parallel. In general, multiple guide RNAs, corresponding to multiple unique genomic loci, are introduced to the plurality of genomic loci. For systems guided by programmable nucleases, the nucleic acid modifying system can then be directed to multiple genomic loci simultaneously. Non-limiting examples of programmable nucleases include CRISPR-Cas polypeptides, Zinc Fingers, TALE nucleases, and Omega systems.

In example embodiments, the set of gRNAs is at least 2 unique gRNAs. A unique gRNA comprises of a gRNA designed to direct a programmable nuclease to a target genomic locus. Two or more gRNAs are unique if they direct a programmable nuclease to different genomic loci. A set of at least 2 unique gRNAs refers to multiple gRNAs (e.g., 2, 3, 4, 5, 10, 100, 1000, 10000, etc.) corresponding to the at least 2 unique gRNAs. For example, a set of 4 gRNAs with 2 unique gRNAs X and Y could correspond to a set of 2X and 2Y or 1X and 3Y or 3X and 1Y. Therefore, in larger examples, any combination of unique gRNAs in a set is considered. In example embodiments, the set of gRNAs comprise of at least 5, 10, 20, 30, 40, 50, 100, 200, 300, 400, 500, 1000 unique gRNAs. In example embodiments, the set of gRNAs comprise between 10 and 1000, 10 and 500, 10 and 250, 10 and 100, 10 and 50, 50 and 1000, 50 and 500, 50 and 250, 50 and 100, 100 and 1000, 100 and 500, 100 and 250, 100 and 200, 250 and 1000, 250 and 500, or 500 and 1000 unique gRNAs. In example embodiments, the set of gRNAs comprise of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140 unique gRNAs.

### Natural Allele Encoding

In example embodiments, the plurality of genomic loci comprise one or more natural alleles which encode information according to an encryption key. Natural alleles comprise an allele which is not edited by a nucleic acid modifying agent and is observed to occur naturally. Natural alleles may be selected based on their location, allele frequency, or combination thereof. For example, a natural allele with an allele frequency of 0.1% may be selected to encode information in an encryption key, wherein the additional alleles, either naturally occurring or modified, also have an allele frequency of 0.1%. Consequently, the encoded information is encrypted in alleles with 0.1% allele frequency.

### Decoding Information

In example embodiments, the method and systems further comprise decoding the information by sequencing the amplified loci and observing an allele frequency at the amplified genomic loci relative to a reference genome. In example embodiments, the methods described herein comprise decoding the information by observing the allele status with allele detection methods. In general, decoding comprises observing, e.g., retrieving/collecting/obtaining the allele status of the genomic locus according to the encryption key and translating the encoded information into its form before the encoding process. For example, the genomic loci according to the encryption key are decoded. The decoded genomic loci (e.g., A, G, T, C, purine, pyrimidine) are then translated based on the encoding scheme (e.g., binary encoding) into the original information.

In an example embodiment, an 8-bit binary encoding scheme is implemented and after decoding the genomic loci, the allele status of the genomic loci corresponding to the encryption key is recorded. The recorded allele corresponds to either a 0 or 1 according to the binary scheme and every 8 genomic loci corresponds to a character of information. Once all the allele statuses are recorded according to the encryption key, the 8-bit binary sequence can be translated to the original information.

A nucleotide at a genomic locus may comprise multiple variants (i.e., allele). In example embodiments, decoding the information comprises sequencing the amplified loci and observing an allele frequency at the amplified genomic loci relative to a reference genome. An allele frequency refers to the number of times (e.g., percentage) a particular variant/allele at a particular genomic locus is observed over one or more genomes relative to a reference genome. The reference genome may be an unmodified genome corresponding to the genome comprising encrypted information or the reference genome is a first modified genome that is then further modified by the methods and systems described herein. An allele frequency of the methods and systems described herein may comprise any percentage between 0.1 and 100. In example embodiments, the allelic frequency of the alleles to the one or more genomes is less than 10%, less than 5%, less than 3%, less than 2%, less than 1%, less than 0.5%, or less than 0.1%. In example embodiments, the allelic frequency of the alleles to the one or more genomes is 0.01%, 0.02%, 0.03%, 0.04%, 0.05%, 0.06%, 0.07%, 0.08%, 0.09%, or 0.1%. In example embodiments, the allelic frequency of the alleles to the one or more genomes is 2%, 3%, 4%, 5%, 6%, 7%, 8%, 9%, or 10%. In example embodiments, the allelic frequency of the alleles to the one or more genomes is 0.2%, 0.3%, 0.4%, 0.5%, 0.6%, 0.7%, 0.8%, 0.9%, or 1%. In example embodiments, the allele frequency is between 0.01% and 10%, between 0.01% and 5%, between 0.01% and 2%, between 0.01% and 1%, between 0.01% and 0.5%, between 0.01% and 0.2%, between 0.01% and 0.1%, between 0.01% and 0.05%, between 0.1% and 10%, between 0.1% and 5%, between 0.1% and 2%, between 0.1% and 1%, between 0.1% and 0.5%, between 1% and 10%, between 1% and 5%, between 1% and 2%. In example embodiments, the allele frequency is at least 1%, at least 2%, at least 3%, at least 5%, at least 10%, at least 15%, at least 20%, at least 25%, at least 33%, at least 50%, at least 66%, at least 75%, at least 100%.

The expected allele frequency may comprise a numerical value that represents the allele frequency of the genomic loci created during the step of editing the plurality of genomic loci by introducing the one or more nucleic acid modifying agents to a cell or population of cells, whereby information is encrypted within the one or more genomes of the cell or population of cells. The expected allele frequency may also comprise a numerical value that represents the observed natural allele frequency of the genomic loci in the absence of man-made and engineered modification of that allele.

### Sequencing Based Decoding

In an example embodiment, methods described herein comprise amplifying polynucleotides comprising the plurality of genomic loci defined by the encryption key; decoding the information by sequencing the amplified loci and observing an allele frequency at the amplified genomic loci relative to a reference genome. Identifying the presence of an edit to the plurality of genomic loci according to the encryption key can be done by any DNA detection method known in the art, including sequencing at least part of a genome of one or more cells.

In certain example embodiments, detection of variants can be done by sequencing. Sequencing can be, for example, whole genome sequencing. In one example embodiment, the disclosure involves high-throughput and/or targeted nucleic acid profiling (for example, sequencing, quantitative reverse transcription polymerase chain reaction, and the like). Any method for detection of mutations from sequencing data may be used. One approach for detection of somatic mutations is to first align both disease (e.g., tumor) and normal reads to a reference genome and then scan the genome and identify mutational events observed in the tumor but not in the matched normal. In example embodiments, the “MuTect” method as described in International Patent Application Publication No. WO2014036167 A1 to Cibulskis et al. is used to detect mutations from alignment data. In example embodiments, the Strelka software (Saunders et al. Bioinformatics. 2012 28, 1811-1817) may be used to detect insertions and deletions in the diseased sample. Polymorphic gene typing may be performed (see, e.g., US20160298185A1).

In example embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. An exemplary sequencing method comprises fragmentation of the genome into millions of molecules or generating complementary DNA (cDNA) fragments, which are size-selected and ligated to adapters. The set of fragments, referred to as a sequencing library, is sequenced to produce a set of reads. Methods for constructing sequencing libraries are known in the art (see, e.g., Head et al., Library construction for next-generation sequencing: Overviews and challenges. Biotechniques. 2014; 56 (2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi: 10.1002/0471142727.mb0422s107 (2014). PMCID: 4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLID platform) or Life Technologies' Ion Torrent platform. Examples of such methods are described in the following references: Margulies et al (Nature 2005 437:376-80); Schneider and Dekker (Nat Biotechnol. 2012 Apr. 10; 30 (4): 326-8); Ronaghi et al. (Analytical Biochemistry 1996 242:84-9); Shendure et al. (Science 2005 309:1728-32); Imelfort et al. (Brief Bioinform. 2009 10:609-18); Fox et al. (Methods Mol. Biol. 2009; 553:79-108); Appleby et al. (Methods Mol. Biol. 2009; 513:19-39); and Morozova et al. (Genomics. 2008 92:255-64), which are incorporated by reference for the general descriptions of the methods and the particular steps of the methods, including all starting products, reagents, and final products for each of the steps.

In certain embodiments, the methods of the present disclosure include whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In certain embodiments, the present disclosure includes whole exome sequencing. Exome sequencing, also known as whole exome sequencing (WES), is a genomic technique for sequencing all of the protein-coding genes in a genome (known as the exome) (see, e.g., Ng et al., 2009, Nature volume 461, pages 272-276). It consists of two steps: the first step is to select only the subset of DNA that encodes proteins. These regions are known as exons—humans have about 180,000 exons, constituting about 1% of the human genome, or approximately 30 million base pairs. The second step is to sequence the exonic DNA using any high-throughput DNA sequencing technology. In certain embodiments, whole exome sequencing is used to determine somatic mutations in genes associated with disease (e.g., cancer mutations).

In certain embodiments, targeted sequencing is used in the present disclosure (see, e.g., Mantere et al., PLOS Genet 12 e1005816 2016; and Carneiro et al. BMC Genomics, 2012 13:375). Targeted gene sequencing panels are useful tools for analyzing specific mutations in a given sample. Focused panels contain a select set of genes or gene regions that have known or suspected associations with the disease or phenotype under study. In certain embodiments, targeted sequencing is used to detect mutations associated with a disease in a subject in need thereof. Targeted sequencing can increase the cost-effectiveness of variant discovery and detection.

Variants may also be detected through hybridization-based methods, including dynamic allele-specific hybridization (DASH), molecular beacons, and SNP microarrays, enzyme-based methods including RFLP, PCR-based, e.g., allelic-specific polymerase chain reaction (AS-PCR), polymerase chain reaction-restriction fragment length polymorphism (PCR-RFLP), multiplex PCR real-time invader assay (mPCR-RETINA), (amplification refractory mutation system (ARMS), Flap endonuclease, primer extension, 5′ nuclease, e.g., Taqman or 5′nuclease allelic discrimination assay, and oligonucleotide ligation assay, and methods such as single strand conformation polymorphism, temperature gradient gel electrophoresis, denaturing high performance liquid chromatography, high-resolution melting of the entire amplicon, use of DNA mismatch-binding proteins, SNPlex, and Surveyor nuclease assay.

### Crispr-Based Decoding

In example embodiments, methods described herein comprise decoding the information by observing the allele status with allele detection methods. In example embodiments, the detection method is a CRISPR-based decoding method.

In example embodiments, the CRISPR-based decoding method comprises a Cas13 variant. In example embodiments, the CRISPR/Cas13-based decoding method is SHERLOCK (Specific High-sensitivity Enzymatic Reporter unLOCKing). SHERLOCK, i.e., one or more CRISPR systems and corresponding reporter constructs, utilizes RNA targeting effectors to provide a robust CRISPR-based diagnostic with attomolar sensitivity. SHERLOCK can detect both DNA and RNA with comparable levels of sensitivity and can differentiate targets from non-targets based on single base pair differences.

For example, the SHERLOCK detection method may generally comprise a two-step process of amplification and detection. During the first step, the nucleic acid sample, either RNA or DNA, is amplified, for example by isothermal amplification. During the second step, the amplified DNA is transcribed into RNA and subsequently incubated with a CRISPR effector, such as C2c2, and a crRNA programmed to detect the presence of the target nucleic acid sequence.

In example embodiments, the plurality of genomic loci is detected by SHERLOCK, wherein the guide molecule of the CRISPR effector is programmed according to the encryption key. SHERLOCK is further described in US Publication Number US20210108267 A1, which is herein incorporated by reference in its entirety.

In example embodiments, the CRISPR-based decoding method comprises a Cas12 variant. In example embodiments, the CRISPR/Cas12-based decoding method is DETECTR (i.e., DNA endonuclease-targeted CRISPR trans reporter). Similar to SHERLOCK, recognition of the target nucleic acid facilitates the cleavage of the quencher bound to the fluorophore thereby producing a fluorescent signal. In example embodiments, the plurality of genomic loci is detected by DETECTR, wherein the guide molecule of the CRISPR effector is programmed according to the encryption key.

Other CRISPR/Cas12-based decoding method include HOLMES (i.e., one-hour low-cost multipurpose highly efficient system), which utilizes either PCR as preamplification or loop-mediated isothermal amplification (LAMP) with a Cas12 protein for attomolar detection. In example embodiments, the plurality of genomic loci is detected by HOLMES, wherein the guide molecule of the CRISPR effector is programmed according to the encryption key.

In example embodiments, the CRISPR-based decoding method comprises a Cas9 variant. In example embodiments, the CRISPR/Cas9-based decoding method is NASBACC (i.e., nucleic acid sequence-based amplification CRISPR) combines Cas9 cleavage for PAM-dependent target detection and nucleic acid sequence-based amplification for the isothermal preamplification. NASBACC relies on a toehold trigger to induce a color change upon detection of the target nucleic acid. In example embodiments, the plurality of genomic loci is detected by NASBACC, wherein the guide molecule of the CRISPR effector is programmed according to the encryption key.

In example embodiments, the CRISPR/Cas9-based decoding method is LEOPARD (i.e., leveraging engineered tracrRNAs and on-target DNAs for parallel RNA detection) is a Cas9-based method which enables the multiplexed detection of different RNA sequences with single-nucleotide specificity. LEOPARD uses modified tracrRNAs to hybridize with cellular RNAs to form non-canonical crRNAs. These non-canonical crRNAa guide the Cas9 complex to DNA targets for detection. In example embodiments, the plurality of genomic loci is detected by LEOPARD, wherein the guide molecule of the CRISPR effector is programmed according to the encryption key.

In example embodiments, multiple genomic loci are detected simultaneously. In example embodiments, SHERLOCK is used to detect multiple genomic loci simultaneously. SHERLOCK detects multiple genomic loci simultaneously by including multiple unique reporter-molecule designs that were specific to the orthogonal collateral-cleavage base preferences such as PsmCas13b (Prevotella sp.), LwaCas13a (Leptotrichia wadeii sp.), CcaCas13b (Capnocytophaga canimorsus Cc5) and AsCas12a (from Acidaminococcus sp.)

In example embodiments, CARMEN (combinatorial arrayed reactions for multiplexed evaluation of nucleic acids) is used to detect multiple genomic loci simultaneously. CARMEN is a CRISPR/Cas13-based decoding method that uses miniaturized reaction volumes to detect multiple genomic loci simultaneously. In particular, a microfabricated chip is loaded with droplet pairs of all possible combinations of amplified nucleic acid target samples and LwaCas13 detection mixes. The droplets comprise of mixing either the samples or detection mixtures with solution-based color code and then emulsified in fluorous oil. The droplet pairs of target sample and detection mixture are merged with an external electric field. Targets are detected by fluorescence generated through Cas13a-triggered reporter cleavage.

For guidance on CRISPR-based detection methods as described herein see Kaminski, M. M., Abudayyeh, O. O., Gootenberg, J. S. et al. CRISPR-based diagnostics. Nat Biomed Eng 5, 643-656 (2021), incorporated herein by reference.

### Mutation Assay-Based Decoding

In example embodiments, methods described herein comprise decoding the information by observing the allele status with allele detection methods. In example embodiments the detection method is SURVEYOR. SURVEYOR technology, in general, comprises four steps. The first step comprises amplifying target DNA from both mutant and reference DNA according to the encryption key, e.g., via PCR. The next step comprises hybridization to form heteroduplexes between mutant and the reference DNA. Then treatment of annealed DNA with SURVEYOR nuclease to cleave heteroduplexes and analysis of digested DNA products using the detection/separation platform of choice.

SURVEYOR is further described in U.S. Pat. Nos. 7,129,075B2 and 7,579,155B2 as well as Qiu, P., et al. Mutation Detection Using Surveyor™ Nuclease. BioTechniques, 2004, 36, 702-707, both of which are hereby incorporated by reference.

In example embodiments, methods described herein comprise decoding the information by observing the allele status with allele detection methods. In example embodiments, the detection method is TAQMAN. TAQMAN technology, in general, comprises DNA amplification and genotype detection, using one or more oligonucleotide probes, one for each allele. The probes are designed to hybridize to a region of the single-stranded DNA according to the encryption key. Fluorogenic reporter dyes are attached to the 5′ ends of the probes, respectively. The fluorescent signal emitted by the reporter dye is suppressed by a quencher at 3′ end of the probes. During amplification, complementary probes attach to the DNA and are degraded by the polymerase's 5′->3′ nuclease activity. Consequently, the probes fluoresce thereby detecting the genomic loci according to the encryption key.

TAQMAN is further described in U.S. Pat. No. 7,052,878 B1 and Koch, W.; et al. TaqMan Systems for Genotyping of Disease-Related Polymorphisms Present in the Gene Encoding Apolipoprotein E. Clinical Chemistry and Laboratory Medicine, 2002, 40, both of which are hereby incorporated by reference.

### Authentication Code

In example embodiments, the encoded information further includes an authentication code defining an expected allele frequency, and wherein the decoding step further comprises comparing the expected allele frequency to an observed allele frequency, wherein an increase in the observed allele frequency relative to the expected allele frequency indicates inauthentic or invalid information. An authentication code (i.e., message authentication code) may comprise a cryptographic checksum, which is used to detect both accidental and intentional modifications to the genome. A cryptographic checksum is a value assigned to encoded information. In example embodiments, the cryptographic checksum is produced by performing multiple mathematical operations to produce a value. Example cryptographic checksum algorithms include, but are not limited to, Message Digest Algorithm 5 (MD5) or Secure Hash Algorithms (SHA). An authentication code may be encrypted into the genome using any encryption method described herein (e.g., symmetric, asymmetric). In example embodiments, the authentication code is the expected allele frequency of the sequenced genomic loci according to the encryption key.

In an example embodiment, the genomic loci comprise an allele frequency of 5% after the encoded information has been encrypted into the genome. Accordingly, the authentication code encodes the information “5”, “5%”, or any variation thereof indicating the allele frequency is 5%. After sequencing the genomic loci according to the authentication key and decoding the encoded information, the authentication code should display 5% (or the variation thereof). Consequently, the allele frequency of the encoded information and authentication code should be 5%, hence the expected allele frequency is 5%.

The observed allele frequency is the measured allele frequency after sequencing the genomic loci according to the encryption key. Continuing with the example embodiment above, genomic loci according to the encryption key is sequenced. The allele frequency of the sequenced genomic loci is measured (i.e., counted) thereby producing the observed allele frequency. If the observed allele frequency is 5%, then the party decoding the encoded information knows the genome and therefore the encoded information has not been modified either accidentally or intentionally. If the observed allele frequency is not 5%, then the party decoding the encoded information knows the genome and therefore the encoded information has been accidentally or intentionally modified.

### Modified Cells

In one aspect, embodiments disclosed herein are directed to biological materials that have been modified as disclosed herein. In example embodiments, a biological material is a modified organism or a modified cell. In example embodiments, the modified cells are from a prokaryote, a eukaryote, or a combination thereof. In example embodiments, the cell is modified to encode information as described herein. For example, the modified cells can include any cell line or primary cell, such as HEK293T cells. The cell(s) may comprise a cell from or in a model non-human organism, for example a model non-human mammal that comprise encrypted genomes encoding information.

In one aspect, an engineered, non-naturally occurring cell, or progeny thereof, wherein the genome of the cell is modified to store encoded information encrypted according to the methods and systems described herein. The modified cells may be generated using the gene editing systems described herein. In example embodiments, the modified cell is a therapeutic cell. Clinical application of CRISPR-Cas9 gene-edited T cells is generally safe and feasible (see, e.g., Lu Y, Xue J, Deng T, et al. Safety and feasibility of CRISPR-edited T cells in patients with refractory non-small-cell lung cancer [published correction appears in Nat Med. 2020 July; 26 (7): 1149]. Nat Med. 2020; 26 (5): 732-740; Lacey S F, Fraietta J A. First Trial of CRISPR-Edited T cells in Lung Cancer. Trends Mol Med. 2020; 26 (8): 713-715; and Zhang X, Cheng C, Sun W, Wang H. Engineering T Cells Using CRISPR/Cas9 for Cancer Therapy. Methods Mol Biol. 2020; 2115:419-433). Immune cells can also be edited ex vivo using Zn Finger proteins (see, e.g., Perez E E, Wang J, Miller J C, et al. Establishment of HIV-1 resistance in CD4+ T cells by genome editing using zinc-finger nucleases. Nat Biotechnol. 2008; 26 (7): 808-816).

To deliver nucleic acid modifying agents to cells for modification, a wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432). Viral vectors may for example include vectors based on HIV, SV40, EBV, HSV or BPV. Despite success with lentiviral delivery, Hendel et al, (Nature Biotechnology 33, 985-989 (2015) doi: 10.1038/nbt.3290) showed the efficiency of editing human T-cells with chemically modified RNA, and direct RNA delivery to T-cells via electroporation.

### Authentication of Biological Material

In one aspect, as described herein, a method of encoding an authentication signature into a biological material, comprising encoding an encrypted verification signature in one or more genomes of the biological material by introducing edits using one or nucleic acid modifying agents at a plurality of genomic loci defined according to an encryption key, whereby measuring the plurality of the genomic loci as defined by the encryption key can be used to identify and/or authenticate the origin or source of the biological material. A verification signature may be any type of information, such as those described herein, associated with the biological material. Information associated with the biological material may be a biological material identification code such as a numerical ID, alphabetical ID, or combination thereof.

In one aspect, as described herein, a method of authenticating a biological material, comprising adding one or more cells to the biological material, the one or more cells comprising information encrypted in a genome(s) of the one or more cells, wherein the encrypted information is used to authenticate the biological material.

In one aspect, as described herein, a method of authenticating a biological material comprising: measuring a set of genomic loci from one or more cells obtained from the biological material and as defined by an encryption key, wherein at least a portion of the cells of the biological material comprises genomes previously edited with one or more nucleic acid modifying agents to encode an authentication code according to the encryption key; wherein an observed allele status at the genomic loci, in combination with the encryption key, are used to decode the authentications signature that confirms an identity of and/or authenticates an origin of the biological material.

Biological authentication is a necessary precaution to prevent cross-contamination, misidentification (e.g., species determination), tampering, or misuse of biological material. The NIH requires authentication of biological material to receive funding grants and the FDA requires authentication of biological material included in investigational new drug applications. Current approaches rely on comparing the genomes of biological material to reference-quality whole genome sequences. The compositions, systems, and methods herein rely on a plurality of genomic loci according to an encryption key to authenticate biological material. Consequently, only the portion of the genome according to the encryption key needs to be sequenced to authenticate the biological material.

In example embodiments, a reproduced biological material comprising an authentication signature or other encrypted information can be authenticated by measuring the allele frequency of the authentication signature or other encrypted information. If the allele frequency is identical to the original biological material, then the reproduced biological material is validated. If the allele frequency is different, then the reproduced biological material has been altered from that of the original biological material.

In example embodiments, compositions, systems, and methods herein can be used for genome encryption in multi-cultures, which includes co-cultures. Multi-cultures attempt to replicate systems of tissues or ecologies to model complex interactions. Genomic encryption can be used to authenticate the process of multi-cultures or track changes to the systems overtime. See e.g., Goers, L.; Freemont, P.; Polizzi, K. M. Co-Culture Systems and Technologies: Taking Synthetic Biology to the next Level. Journal of The Royal Society Interface, 2014, 11, 20140065 and Diender, M.; Parera Olm, I.; Sousa, D. Z. Synthetic Co-Cultures: Novel Avenues for Bio-Based Processes. Current Opinion in Biotechnology, 2021, 67, 72-79.

In example embodiments, compositions, systems, and methods herein can be used for genome encryption in cell-based sensors, including cell-based screens. Cell-based sensors are used, for example, to detect changes in the environment (e.g., sample toxicity or soil conditions) or pharmacology (e.g., drug screening). Cell-based sensors use transduction/detection methods such as electrical cell-substrate impedance sensing (ECIS), light addressable potentiometric sensor, and fluorescent imaging. The engineered cells for cell-based sensing may comprise authentication signatures or otherwise encrypted information. See e.g., Gheorghiu, M. A Short Review on Cell-Based Biosensing: Challenges and Breakthroughs in Biomedical Analysis. The Journal of Biomedical Research, 2021, 35, 255.

In example embodiments, compositions, systems, and methods herein can be used for genome encryption and/or authentication in cell-based models, such as disease and drug models including Organ-on-a-Chip. See e.g., Ma, C.; et al. Organ-on-a-Chip: A New Paradigm for Drug Development. Trends in Pharmacological Sciences, 2021, 42, 119-133, Wu, Q.; et al. Organ-on-a-Chip: Recent Breakthroughs and Future Prospects. BioMedical Engineering OnLine, 2020, 19.

### Cell-Based Therapies

In example embodiments, wherein the biological material is a modified organism or a modified cell, such as those described elsewhere herein, the modified cell may comprise a therapeutic cell. In some embodiments, the therapeutic cells can be used in cell-based therapies. A method of a cell therapy generally includes administering, using a suitable method or technique, a modified cell or cell population (or a pharmaceutical formulation thereof) to a subject in need thereof. It will be appreciated that the cells can be autologous or allogeneic. In some embodiments, the modified cells are allogeneic and include modifications so as to reduce the recipient's immune or other response to the modified cells to increase efficacy of the therapy. In some embodiments, where the modified cell(s) are allogeneic, the method can comprise administering the cells with one or more protective biomaterials that are capable of shielding the allogenic cells from the recipient's immune system.

Cell-based therapies may include regenerative and tissue and/or organ replacement therapies. For example, replacement therapies may include adoptive cell therapies (ACT). ACT can be categorized into three groups: tumor-infiltrating lymphocytes (TIL), T cell receptor (TCR) gene therapy, and chimeric antigen receptor (CAR) modified T cells. Other immune cell types, such as natural killer cells, are also being investigated as a basis for cell therapy. See e.g., Rohaan, M. W.; et al. Adoptive Cellular Therapies: The Current Landscape. Virchows Archiv, 2018, 474, 449-461, Weber, E. W.; et al. The Emerging Landscape of Immune Cell Therapies. Cell, 2020, 181, 46-62, and Perez, C.; et al. Off-the-Shelf Allogeneic T Cell Therapies for Cancer: Opportunities and Challenges Using Naturally Occurring “Universal” Donor T Cells. Frontiers in Immunology, 2020, 11.

Cell-based replacement therapies may also comprise delivering keratinocytes, fibroblasts, bone marrow, and/or adipose tissue-derived mesenchymal stem cells to improve chronic wound healing by delivery of different cytokines, chemokines, and growth factors. See e.g., Domaszewska-Szostek, A.; et al. Cell-Based Therapies for Chronic Wounds Tested in Clinical Studies. Annals of Plastic Surgery, 2019, 83, e96-e109. Cell-based replacement therapies may also comprise replacement of beta, islet, CNS, neuron, tissue, or stem cell replacement therapies. See e.g., Brasile, L.; Stubenitsky, B. Will Cell Therapies Provide the Solution for the Shortage of Transplantable Organs? Current Opinion in Organ Transplantation, 2019, 24, 568-573, Yamanaka, S. Pluripotent Stem Cell-Based Cell Therapy—Promise and Challenges. Cell Stem Cell, 2020, 27, 523-531, and/or Madrid, M.; et al. Autologous Induced Pluripotent Stem Cell-Based Cell Therapies: Promise, Progress, and Challenges. Current Protocols, 2021, 1.

Cell-based regenerative and replacement therapies comprise engineering biological structures, such as tissue; organs; or a portion thereof, via in vitro fabrication. See e.g., Langer, R.; Vacanti, J. Advances in Tissue Engineering. Journal of Pediatric Surgery, 2016, 51, 8-12, Bakhshandeh, B.; et al. Tissue Engineering; Strategies, Tissues, and Biomaterials. Biotechnology and Genetic Engineering Reviews, 2017, 33, 144-172. Shafiee, A.; Atala, A. Tissue Engineering: Toward a New Era of Medicine. Annual Review of Medicine, 2017, 68, 29-40.

Cell-based therapies may also comprise administration of engineered cells for delivery of substances, e.g., drugs such as antibiotics, vaccines, and antibodies, for example where the cells are engineered via therapeutic bioreactors.

Cell-based therapies may also comprise administering engineered or otherwise modified microbiomes. Engineered microbiomes are used directly as treatment or preventing adverse effects from other therapies. For example, direct treatments using engineered microbiomes include fecal microbiota transplant, prebiotics, probiotics, synbiotics and synthetic microbes. Example preventative measures using microbes include drug reactivation (e.g., β-glucuronidases), drug deactivation (e.g., tyrosine decarboxylase), or toxic byproducts. See e.g., Khan, S.; Hauptman, R.; Kelly, L. Engineering the Microbiome to Prevent Adverse Events: Challenges and Opportunities. Annual Review of Pharmacology and Toxicology, 2021, 61, 159-179.

### Xenotransplantation

The present disclosure also contemplates use of the compositions, systems, and methods described herein for genome encryption in modified tissues for transplantation. Xenotransplantation comprises, for example, the use of RNA-guided DNA nucleases to knockout, knockdown or disrupt selected genes in an animal, such as a transgenic pig (such as the human heme oxygenase-1 transgenic pig line) or, for example, by disrupting expression of genes that encode epitopes recognized by the human immune system, i.e. xenoantigen genes. Candidate porcine genes for disruption may for example include α(1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Xenotransplantation also relates to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (New insights into repeat instability: role of RNA·DNA hybrids. McIvor E I, Polak U, Napierala M. RNA Biol. 2010 September-October; 7 (5): 551-8). Effector protein systems may be harnessed to correct these defects of genomic instability.

Xenotransplantation may also relate to correcting defects associated with a wide range of genetic diseases which are further described on the website of the National Institutes of Health under subsection the topic Genetic Disorders (website at health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

### Agricultural Products

In example embodiments, the biological material is a modified organism or a modified cell, where the modified organism is a modified plant. In example embodiments, the method further comprises adding one or more cells to the biological material, the one or more cells comprising information encrypted in a genome(s) of the one or more cells, wherein the encrypted information is used to authenticate the biological material. The compositions, systems, and methods described herein can be used to perform gene or genome interrogation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fungus genome. There can accordingly be improved production of plants, new plants with new combinations of traits or characteristics or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to authenticate/monitor desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to authenticate/monitor endogenous genes or to authenticate/monitor their expression without the permanent introduction into the genome of any foreign gene.

In one embodiment, genome editing in plants or where RNAi or similar genome editing techniques have been used previously are used for genomic encryption; see, e.g., Nekrasov, “Plant genome editing made easy: targeted mutagenesis in model and crop plants using the CRISPR-Cas system,” Plant Methods 2013, 9:39 (doi: 10.1186/1746-4811-9-39); Brooks, “Efficient gene editing in tomato in the first generation using the CRISPR-Cas9 system,” Plant Physiology September 2014 pp 114247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi: 10.1038/cr.2013.114; published online 20 Aug. 2013; Xie, “RNA-guided genome editing in plants using a CRISPR-Cas system,” Mol Plant. 2013 November; 6 (6): 1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al., “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061-Agrobacterium-Mediated Plant Transformation Method; U.S. Pat. No. 7,868,149—Plant Genome Sequences and Uses Thereof and US 2009/0100536-Transgenic Plants with Enhanced Agronomic Traits, Morrell et al “Crop genomics: advances and applications,” Nat Rev Genet. 2011 Dec. 29; 13(2): 85-96, all the contents and disclosure of each of which are herein incorporated by reference in their entirety. Aspects of utilizing the compositions, systems, and methods may be analogous to the use of the composition in plants, and mention is made of the University of Arizona website “CRISPR-PLANT” (genome.arizona.edu/crispr/) (supported by Penn State and AGI).

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The compositions, systems, and methods may be used for screening genes (e.g., endogenous, mutations) of interest. In some examples, genes of interest include those encoding enzymes involved in the production of a component of added nutritional value or generally genes affecting agronomic traits of interest, across species, phyla, and plant kingdom. By selectively targeting e.g. genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present disclosure encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K C, et al., Codon Optimization to Enhance Expression Yields Insights into Chloroplast Translation, Plant Physiol. 2016 September; 172 (1): 62-77.

The components (e.g., CRISPR-Cas polypeptide nuclease) in the compositions and systems may further comprise one or more functional domains described herein. In some examples, the functional domains may be an exonuclease. Such exonuclease may increase the efficiency of the Cas5-HNH polypeptide nuclease' function, e.g., mutagenesis efficiency. An example of the functional domain is Trex2, as described in Weiss T et al., www.biorxiv.org/content/10.1101/2020.04.11.037572v1, doi doi.org/10.1101/2020.04.11.037572.

In example embodiments, compositions, systems, and methods herein can be used for genome encryption in engineered or otherwise modified microbiome. Plant associated microbes (e.g., phytomicrobiomes) are engineered to enhance plant growth-promoting traits, such as yield or resilience. See e.g., Ke, J.; Wang, B.; Yoshikuni, Y. Microbiome Engineering: Synthetic Biology of Plant-Associated Microbiomes in Sustainable Agriculture. Trends in Biotechnology, 2021, 39, 244-261, Arif, I.; Batool, M.; Schenk, P. M. Plant Microbiome Engineering: Expected Benefits for Improved Crop Growth and Resilience. Trends in Biotechnology, 2020, 38, 1385-1396, Foo, J. L.; et al. Microbiome Engineering: Current Applications and Its Future. Biotechnology Journal, 2017, 12, 1600099, and Bano, S.; WU, X.; Zhang, X. Towards Sustainable Agriculture: Rhizosphere Microbiome Engineering. Applied Microbiology and Biotechnology, 2021, 105, 7141-7160.

In example embodiments, compositions, systems, and methods herein can be used for genome encryption in food products. For example, cultivated meat is produced in vitro and the cells sourced for this process is an important aspect. Therefore, authenticating cell lines with genome encryption can add a level of safety and security to the process. See e.g., Reiss, J.; Robertson, S.; Suzuki, M. Cell Sources for Cultivated Meat: Applications and Considerations throughout the Production Workflow. International Journal of Molecular Sciences, 2021, 22, 7513 and Pajčin, I.; et al. Bioengineering Outlook on Cultivated Meat Production. Micromachines, 2022, 13, 402.

In example embodiments, compositions, systems, and methods herein can be used for genome encryption in agricultural-based cell bioreactors. These cell bioreactors may be used to create industrial chemicals such as fuels, in the food industry such as brewing, and cosmetics. See e.g., Eibl, R.; et al. Plant Cell Culture Technology in the Cosmetics and Food Industries: Current State and Future Trends. Applied Microbiology and Biotechnology, 2018, 102, 8661-8675.

### Examples of Plants

The compositions, systems, and methods herein can be used for genome encryption in essentially any plant. A wide variety of plants and plant cell systems may encrypt information. In general, the term “plant” relates to any various photosynthetic, eukaryotic, unicellular or multicellular organisms of the kingdom Plantae characteristically growing by cell division, containing chloroplasts, and having cell walls comprising of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna, and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In one embodiment, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of amphora, anabaena, anikstrodesmis, botryococcus, chaetoceros, chlamydomonas, chlorella, chlorococcum, cyclotella, cylindrotheca, dunaliella, emiliana, euglena, hematococcus, isochrysis, monochrysis, monoraphidium, nannochloris, nannnochloropsis, navicula, nephrochloris, nephroselmis, nitzschia, nodularia, nostoc, oochromonas, oocystis, oscillartoria, pavlova, phaeodactylum, playtmonas, pleurochrysis, porhyra, pseudoanabaena, pyramimonas, stichococcus, synechococcus, synechocystis, tetraselmis, thalassiosira, and trichodesmium.

### Specific Plant Organelles

The compositions and systems herein may comprise encrypting information into the genome, or a portion thereof, in a specific plant organelle. In an example embodiment, it is envisaged that the compositions and systems are used to specifically encrypt information into chloroplast genes, or a portion thereof.

Plants with Desired Traits

The compositions, systems, and methods herein may be used to encrypt information into the genome, or a portion thereof, into plants with desired traits. This approach allows monitoring of the plants with desired traits. Monitoring may include identifying genetic alterations to the plant with desired traits or ownership of a plant with desired traits.

### Encryption of Polyploid Plants

The compositions, systems, and methods may be used to encrypt information into the genome, or a portion thereof, of polyploid plants. Polyploid plants carry duplicate copies of their genomes (e.g. as many as six, such as in wheat). In some cases, the compositions, systems, and methods may be/can be multiplexed to affect all copies of a gene, or to target dozens of genes at once. For instance, the compositions, systems, and methods may be used to simultaneously ensure encryption in different genes responsible for suppressing defenses against a disease. The modification may be simultaneous suppression the expression of the TaMLO-Al, TaMLO-Bl and TaMLO-Dl nucleic acid sequence in a wheat plant cell and regenerating a wheat plant therefrom, in order to ensure that the wheat plant is resistant to powdery mildew (e.g., as described in WO2015109752).

### Plant Cultures and Regeneration

In one embodiment, the modified plants or plant cells may be cultured to regenerate a whole plant which possesses the genome encrypted information. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

### Detecting Modifications in the Plant Genome-Selectable Markers

When the compositions, systems, and methods are used to encrypt information into the genome, or a portion thereof, of a plant, suitable methods may be used to confirm and detect the modification made in the plant. In some examples, when a variety of modifications are made, one or more desired modifications or traits resulting from the modifications may be selected and detected. The detection and confirmation may be performed by biochemical and molecular biology techniques such as those described herein.

Genome encryption may be used for selecting, monitoring, isolating cells and plants with desired modifications and traits. Genome encryption can confer positive or negative selection and is conditional or non-conditional on the presence of external substrates.

### Applications in Fungi

The compositions, systems, and methods described herein can be used to encrypt information into the genome, or a portion thereof, in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells include yeasts, molds, and filamentous fungi.

In one embodiment, the fungal cell is a yeast cell. A yeast cell refers to any fungal cell within the phyla Ascomycota and Basidiomycota. Examples of yeasts include budding yeast, fission yeast, and mold, S. cerervisiae, Kluyveromyces marxianus, Issatchenkia orientalis, Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In one embodiment, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In one embodiment, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In one embodiment, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In some examples, the abundance of guide RNA may more often be a rate-limiting component in genome engineering of polyploid cells than in haploid cells, and thus the methods using the composition described herein may take advantage of using certain fungal cell types.

In one embodiment, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In one embodiment, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

### Applications in Non-Human Animals

The compositions, systems, and methods may be used to authenticate/monitor non-human animals. In one embodiment, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include, but are not limited to, horses, goats, sheep, swine, cattle, llamas, alpacas, and birds, e.g., chickens, turkeys, ducks, and geese. The animals may be non-human primates, including but not limited to, baboons, capuchin monkeys, chimpanzees, lemurs, macaques, marmosets, tamarins, spider monkeys, squirrel monkeys, and vervet monkeys. Examples of pets include, but are not limited to, dogs, cats, horses, wolves, rabbits, ferrets, gerbils, hamsters, chinchillas, fancy rats, guinea pigs, canaries, parakeets, and parrots.

### Other Example Uses

In one aspect, a method of encryption comprising: mixing two or more sets of genomes, wherein the sets of genomes are mixed according to one or more encryption keys, wherein the one or more encryption keys link encoded information to a genomic loci thereby creating a set of genomic loci coordinates that hold the encoded information and defining an allele status for each genomic loci in the set of genomic loci coordinates.

In example embodiments, the genomes are not modified by a nucleic acid modifying agent before the genomes are mixed. In example embodiments, only one set of genomes, or a portion of one set of genomes, are modified by one or more nucleic acid modifying agents before the genomes are mixed. In example embodiments, any number of sets of genomes (e.g., 1%, 2%, 5%, 10%, 25%, 50%, 75%, 100%), or portion thereof, are modified by one or more nucleic acid modifying agents before the genomes are mixed.

In one aspect, a method of encryption comprising: mixing two or more cells or two or more population of cells, the cells or population of cells are mixed according to one or more encryption keys, wherein the one or more encryption keys link encoded information to a genomic loci thereby creating a set of genomic loci coordinates that hold the encoded information and defining an allele status and allele frequency for each genomic loci in the set of genomic loci coordinates, whereby information is encrypted within the one or more genomes of the cell or population of cells.

In example embodiments, the genomes of the cells are not modified by a nucleic acid modifying agent before the cells are mixed. In example embodiments, only one cell or one population of cells, or a portion of the one population of cells, are modified by one or more nucleic acid modifying agents before the cells are mixed. In example embodiments, any number of cells or population of cells (e.g., 1%, 2%, 5%, 10%, 25%, 50%, 75%, 100%) are modified by one or more nucleic acid modifying agents before the cells are mixed.

In one aspect, a method of authenticating a biological material comprising: measuring a set of genomic loci from one or more cells obtained from the biological material and as defined by one or more encryption keys, wherein at least a portion of the cells of the biological material comprises genomes mixed to encode an authentication code according to the one or more encryption keys; wherein an observed allele status at the genomic loci, in combination with the one or more encryption keys, are used to decode the authentications signature that confirms an identity of and/or authenticates an origin of the biological material.

Further embodiments are illustrated in the following Examples which are given for illustrative purposes only and are not intended to limit the scope of the invention.

### EXAMPLES

### Example 1—Cryptography in Living Cells

### Results

For encoding messages, Applicants created a key that links the positions of characters of the encoded message to genomic loci at which corresponding mutations can then be installed using genome engineering (FIG. 1A). Applicants chose an implementation where mutations correspond to binary (‘1’) bits, and reference bases to binary ‘0’ bits (FIG. 1B). Applicants implemented this encoding scheme using the Cas9-based base editor AncBE4max (Koblan et al. 2018), which performs gRNA programmable deamination of cytosines into uridines. Uridine base pairs as thymine and edited sites are thus identified as reference cytosines converted into thymines or, on the opposite strand, guanines converted into adenines. A recipient with the key can sequence the amplicons at high coverage per position using high throughput sequencing, and analyze the read data to call if bases are edited at the positions of interest (FIG. 1C).

Here, Applicants sought to scalably install edits in parallel with AncBE4max through massively parallel editing of a population of cells. To enable parallel writing for message encoding, Applicants first screened for a set of gRNAs which allows robust encoding of messages in a single transfection. First, Applicants designed ˜400 gRNAs by selecting random coordinates of the human genome across all chromosomes and searching for cytosines/guanosines which can be targeted using AncBE4max, i.e. bases that are located in proximity to the required PAM site and within the editing window of the base editor. gRNAs were screened for editing efficiency in pools of 48 gRNAs (FIG. 4), and a set of 110 gRNAs with target sites amenable to PCR amplification high editing efficiencies (>0.5% editing) and low background editing were selected for encoding messages. Applicants aimed for a design where all edits can be installed in one round of transfection. As Applicants found editing rates to be inversely proportional to the number of gRNAs at high gRNA batch sizes (FIG. 5), Applicants reasoned that 110 sites of which ˜50% are split in a random binary message would enable editing frequencies that allow robust decoding.

For analyzing the sensitivity and specificity of the gRNAs selected for message encoding and decoding, 110 gRNAs were split into two batches where the two batches contain all even or uneven numbered positions of the binary messages, respectively (FIG. 1D). Analyzing editing rates of all sites in HEK293 Ts showed a true positive rate of 96.36% and a true negative rate of 96.36% at a threshold of 0.1% editing, (FIG. 1E). The area under the curve for true positive and true negative rates at different editing thresholds was calculated to be 0.980, and editing rates are highly correlated between biological replicates with a Pearson correlation coefficient of 0.902 (FIG. 1F). These results suggest highly parallelized base editing in mammalian genomes is achievable and is a facile method for information encoding that allows for reliable retrieval of information.

Applicants next set out to determine the difficulty of breaking the encryption for an adversary who does not have access to the key. In contrast to an intended recipient, an adversary is not able to amplify select sites but needs to search for mutations over the full genome. This is achieved by whole genome sequencing and subsequent variant calling in order to identify mutations (FIG. 2A). A mutated site corresponding to a flipped can be called a variant (true positive, TP) or might go undetected (false negative, FN) for reasons such as insufficient coverage or low editing frequency. On the other hand, sites in the genome might be wrongly called (false positive, FP) due to reasons including sequencing error, SNPs, or artifacts occurring during library preparation.

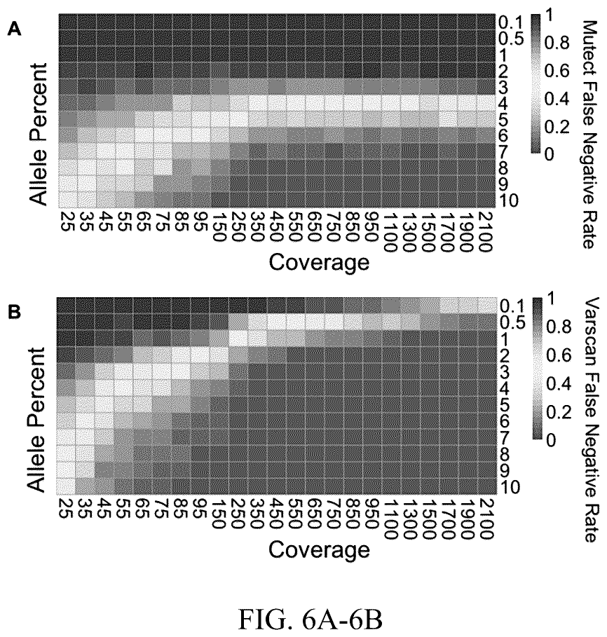

In order to determine how false negative rate and false positive rate influence the difficulty for an adversary to break the encryption, Applicants developed a simulation framework in which Applicants introduced synthetic edits into a published human deep sequencing data set (Schiroli et al. 2019) sequenced on an Illumina NGS platform, and examine the performance of two commonly used variant callers, Mutect and VarScan2. Applicants determined the false negative rates for different allele frequencies and sequence coverages and observed that the false negative rate is inversely proportional to the allele frequency of the edit and to sequencing coverage (FIG. 2B and FIG. 6). While VarScan2 and Mutect had comparable performances at high allele frequencies, only VarScan2 was able to successfully detect variants at allele frequencies below ˜2% (FIG. 6) and Applicants thus decided to continue the analyses with VarScan2. When modeling the false positive rate, Applicants observed that the rate depends on the variant caller sensitivity and thus indirectly on the minimum allele frequency of the edits (FIG. 2B), as the sensitivity would need to be set to a value that allows detection of true edits. At a sensitivity of 0.1%, the false positive rate corresponds to ˜4% (millions of FP sites across a human genome). These data suggest that an adversary would not be able to discern index sites by observing a single message.

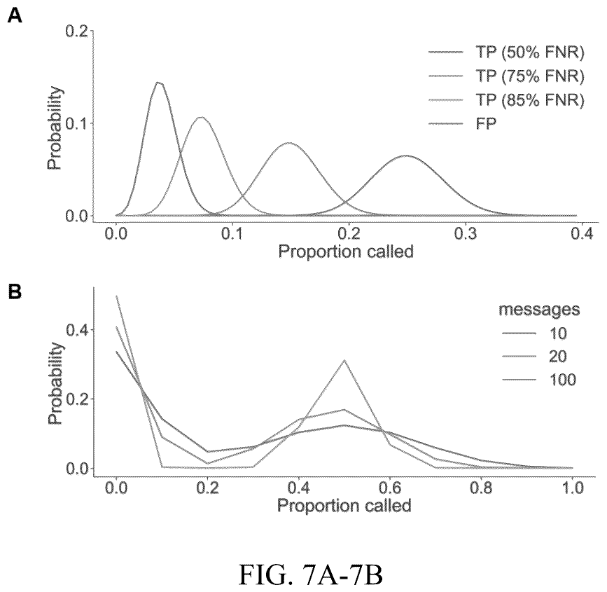

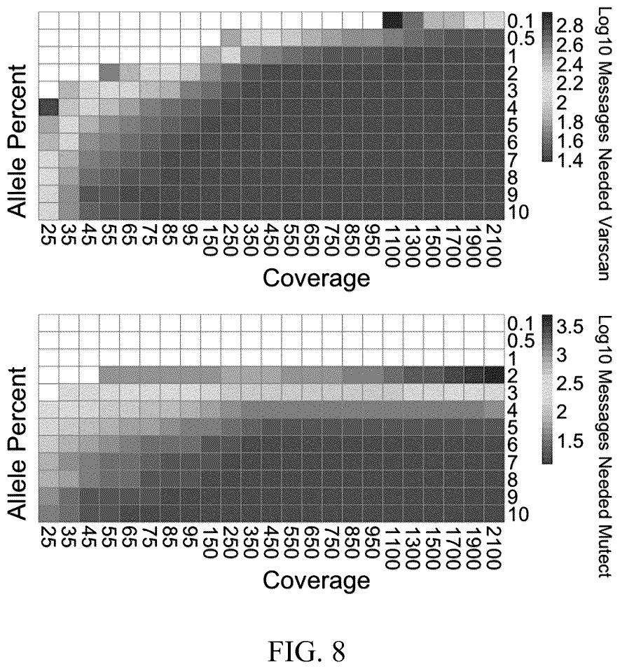

Applicants next determined the number of messages needed to reveal the key indices. Applicants reasoned that an adversary would be increasingly able to observe true index sites and distinguish between true key sites and false positives when observing more messages, as true positives would be called at a higher fraction of messages compared to false positives, which are randomly distributed in the genome (FIG. 2C, FIG. 7). Given infinite coverage per message, Applicants determined the minimum number of messages required to break the code to be 24 messages at an editing frequency of 0.1% and a decreasing number of messages at higher allele frequencies (FIG. 2E).

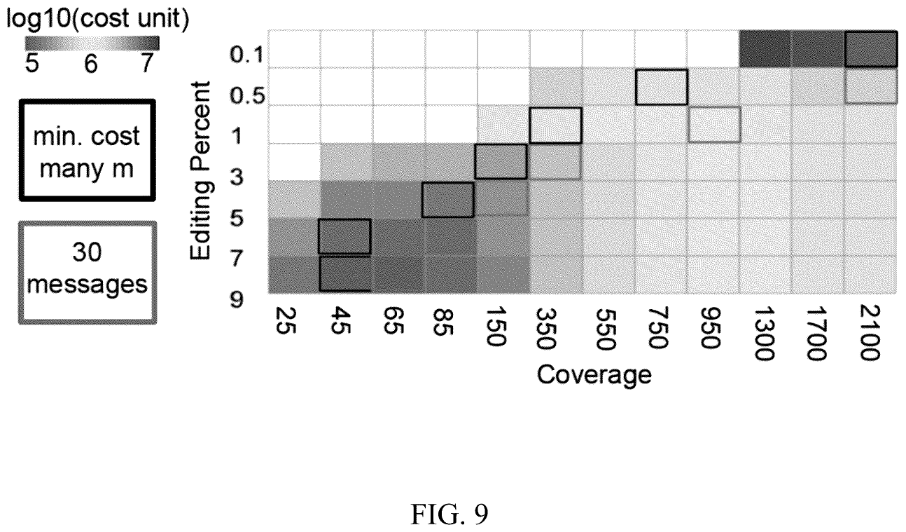

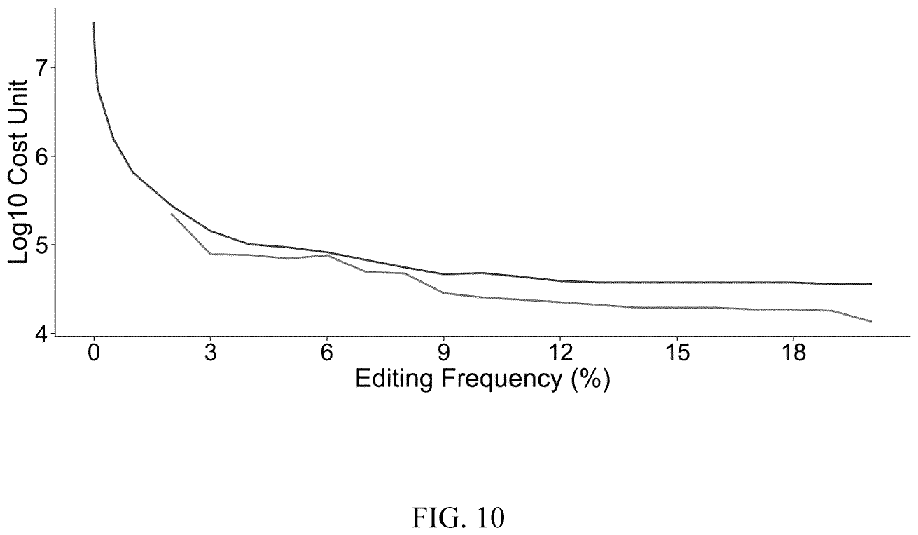

Next, assuming that the adversary can observe many messages using the same key, Applicants sought to examine the adversary cost as a function of allele frequency. Applicants calculated the total cost for two scenarios; in the first scenario an adversary can freely vary sequencing coverage and the number of observed messages (FIG. 9), while in the second scenario an adversary sets out to break the code in less than 30 messages, analogous to a more realistic situation in which an adversary is message limited (FIG. 2D). Applicants compared this cost to the cost of a recipient who has access to the key (FIG. 2E, FIG. 10) and found that the cost of breaking the code, i.e. determining 90% key indices among a tolerable number of false positive hits, is ˜10\hat{ }5 higher than for a recipient at 5% editing. This cost increases non-linearly at lower allele frequencies: At an editing frequency of 0.5% which is well above the detection limit of 0.1%, the cost difference between adversary and recipient is greater than 10\hat{ }6, which increases up to 5×10\hat{ }6 at 0.1%.

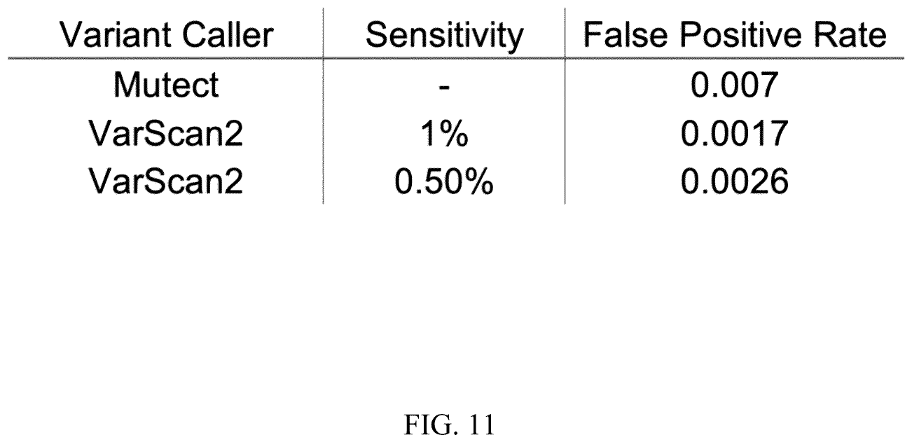

In order to experimentally test the detection of flipped bits from an adversary's perspective, Applicants sought to retrieve mutations with and without known coordinates. Applicants designed 24 gRNAs that introduced non-coding mutations in the human exome when used with AncBE4max, and transfected them into HEK293 Ts. Editing was confirmed using amplicon sequencing to obtain ground truth editing frequencies. Whole exome sequencing was performed at a coverage of >1000× and variant calling was performed. For mutations with editing rates above 1%, Mutect and VarScan with a sensitivity threshold of 1% both allowed detection of 4 out of 5, while decreasing the sensitivity threshold of VarScan to 0.5% resulted in detection of 5 out of 5 mutations. For mutations ranging from 0.3% to 1%, 1 out of 5 mutations was detected for all three Variant Calling modalities. Applicants also calculated the false positive rates by counting all SNPs across the exome and found that false positives were called at a rate of 0.007 for Mutect and 0.0017 and 0.0026 for VarScan with a 1% and 0.5% sensitivity threshold (FIG. 11). These results suggest that even at very high coverage rates, detection of low-frequency mutations remains difficult and a high number of false positives further confounds detection of the key.

Taken together, the results show that revealing key indices is asymmetrically difficult for an adversary, and requires the observation of multiple messages even with infinite sequencing budgets.

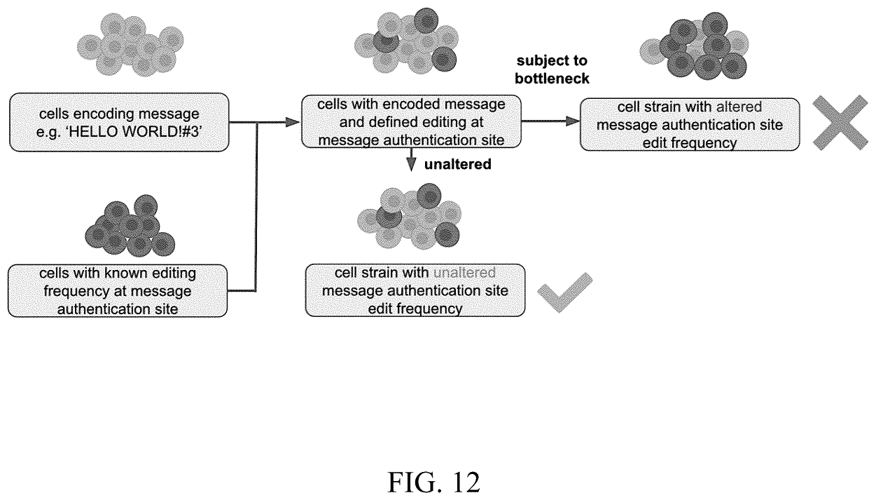

In many cryptographic schemes, a method authentication code is used to validate the authenticity of data. Here, Applicants again leverage allelic frequency within a population of cells by creating an edit at a defined editing percentage at the message authentication site (FIG. 12). The editing percentage is then encoded as part of the message, and is thus cryptographically secured and only by decoding the message can the authentication be verified. A genomic modification that is installed by the sender at the desired editing percentage can be used by a receiver to gain information about the integrity of the cell strain: The editing frequency may change due to genetic drift when a population of cells is subjected to a genomic bottleneck such as during a selection step upon genomic alteration, while it may remain stable in the case of an unmodified strain (FIG. 3A).

In order to verify that editing frequencies remain stable under regular growth conditions while being shifted when the strain is perturbed, Applicants created a cell line with silent mutations. After three days in culture, cells were bottlenecked to 50, 100, 500 and 1000 cells, or maintained under regular conditions for 15 days where cells were passaged every three days. Applicants observed that edits remain more stable under regular passage conditions, where the highest absolute change in editing frequency was by a factor of 2.78 at any of the passages (FIG. 3B). In contrast, for the cell population bottlenecked at 500 cells, the highest absolute change was 5.18-fold. Applicants observed that subjecting cell populations to more stringent bottlenecks led to a larger disruption of editing frequencies (FIG. 3C). In addition, Applicants observe that 8/20 and 6/20 edited sites were no longer detected after the population was bottlenecked to 50 and 100 cells, respectively (FIG. 3C). Applicants next calculated the fraction of sites for which the editing percentage is perturbed over different log fold change thresholds (FIG. 3d). These results suggest that comparing editing sites to initial editing frequencies carries information about whether a strain has been subjected to bottlenecks such as would occur when strains are subjected to selection steps or sorting steps when introducing genetic modifications.

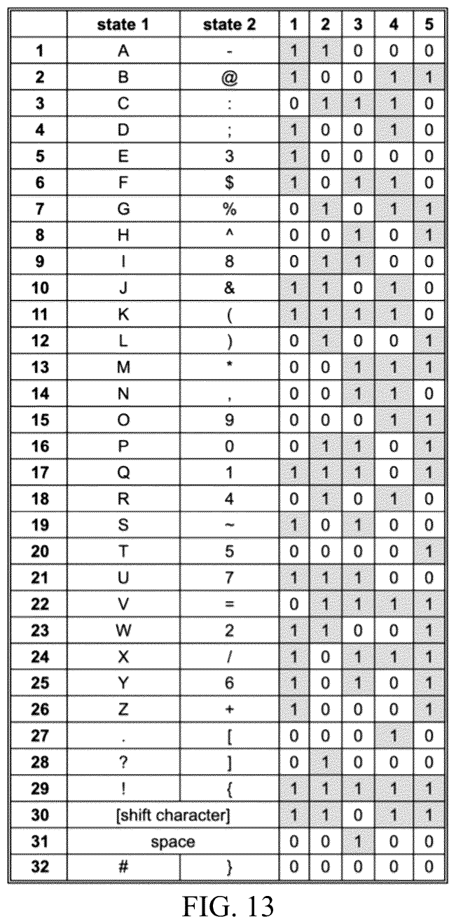

Finally, Applicants demonstrate the encoding of messages including the message authentication value. Applicants employed a modified version of the five-bit International Telegraph Alphabet no. 2 (ITA2; FIG. 13) for converting text to binary. Using 5-bit encoding, 110 gRNA sites are able to encode a message up to 22 characters in length. Three messages were selected for encoding: ‘HELLO WORLD!#3’, ‘WHAT HATH GOD WROUGHT?’ and ‘221B BAKER STREET #2’, with digits at the end of the message representing the editing percentage Applicants aimed at installing at the message authentication site. The messages were encoded into binary and for each of the messages, gRNAs corresponding to ‘1’ were transfected into HEK293T while ‘0’ indices were excluded from the gRNA pool. Finally, an edit at the message authentication site was added at a defined editing percentage.

Applicants read the messages using amplicon sequencing and observed that for each of the three messages less than 3% of all bits were misclassified (FIG. 3E). For the anti-modification site, Applicants achieved editing values that are within a ˜10% of the desired editing frequencies (FIG. 3F). These results suggest that the encoding scheme enables robust encoding and decoding of messages, and that cell strains with anti-modification edit can be mixed in to achieve defined editing frequencies.

### Discussion

Here, Applicants demonstrate a cryptographic system for information encoded in the genome of living organisms. The scheme is based on the asymmetric cost of detecting genomic mutations when genomic coordinates of potential variation are known to the intended recipient, but unknown to an adversary who is trying to crack the message, and is thus based purely on the properties of DNA sequencing and sequence analysis, especially in the context of low allelic frequencies. Applicants demonstrate that genomically encoded information can be decoded by a recipient with access to the key, i.e. knowledge of the genomic coordinates. Applicants also show, through detailed computational simulation and experimental data, that an adversary who does not have access to the key cannot break the code until a certain number of messages has been observed. After that number of messages, an adversary is theoretically able to break the key at a considerably higher cost than a recipient with the key. This asymmetric cost function resembles current computational encryption algorithms.

Encrypting a message in living cells allows the confidential transfer of messages. Applicants show that base editors are suitable for introducing targeted point mutations in mammalian cells in a highly parallelized way. Like other Cas9-based editing approaches, they have the advantage of being easily programmable and multiplexable enabling efficient encoding of messages in pooled gRNA transfections. Here, Applicants show the encoding of up to 110-bit messages. Applicants anticipate that longer messages can be encoded either in a single transfection by screening for a larger number of gRNAs with high editing efficiencies, or through iterative rounds of transfections.

The current implementation uses cytosine base editors which require the presence of an NGG PAM site at a certain distance from a target cytosine. While an adversary could use this information to limit his search space, Applicants expect that this potential limitation can be overcome. In order to overcome the reliance on PAM sites for editing while maintaining the programmability of Cas9, newer evolved BEs with relaxed or altered PAM site requirements (Walton et al. 2020; Chatterjee et al. 2020) can be employed. In addition, the method can be adapted to include other genome editors and types of mutations: Adenosine Base editors (Gaudelli et al. 2017) can be used to introduce adenine to guanine and thymine to cytosine mutations, or prime editors (Anzalone et al. 2019) can be used for introducing any type of point mutation.

Applicants further demonstrate use of the encoding scheme to verify the integrity of a population of cells. In addition to verifying the sender of the message/cells, the message authentication step is further able to validate whether a cell line is in its original genomic state or has been genetically modified because of the genetic drift in edited alleles that occurs when a population is subjected to a bottleneck such as during selection steps. While Applicants have demonstrated this in a human cell line, the approach is applicable to other living organisms which are amenable to parallelized genome editing including bacteria and multicellular organisms.

As the drop in sequencing cost is expected to continue, Applicants anticipate the cost that is required for an adversary to break the key to also decrease in the future. As the cost function between the adversary and recipient cost is highly asymmetrical at low allele frequencies, Applicants anticipate that encoding messages at lower allele frequencies will be able to scale with a drop in sequence cost. GSE is orthogonal and can be combined with cryptographic approaches relying on computational difficulty. In addition, the scheme can be extended to include other information security concepts, such as ‘winnowing and chaffing’, where additional genomic edit sites which are not used as key sites are added which increases the difficulty of identifying correct sites (Rivest).

Lastly, while Applicants implement symmetric key encoding here, an extendable asymmetric key scheme could be implemented in which the public key is a set of base editors complexed with gRNAs, and the private key is the set of indices. Analogous to computational public/private key systems, a shareable public key improves security by enabling anyone to encode a message while the private key would be required to read the message. While in this scheme, an adversary sequencing gRNAs from the public key might be able to break the code, alternative approaches including genome editors that function without gRNAs like TALEN or ZFN-based editing approaches can be investigated to overcome this practical limitation.

GSE is a generalized biological cryptographic scheme for message exchange. Genomic encryption schemes are going to be required as DNA is becoming a crucial medium for information exchange. GSE is orthogonal to existing cryptographic approaches and as it does not rely on computational difficulty, it is not affected by increases in computing performance including the emergence of quantum computers. GSE can be broadly applied as a signature for living biological materials. This allows genetically modified strains to be cryptographically signed and authenticated over generations as genomic edits are propagated. As genome engineering for cellular therapies and GMOs become more widespread, Applicants anticipate the approaches proposed here to be useful for securing and authenticating biological materials and supply chains.

### Materials and Methods

gRNA Design

For designing gRNAs, random genome indices were retrieved using bedtools (bedtools version 2.27.1) running the command ‘bedtools random’ on the human reference genome hg38. Corresponding fasta sequences were extracted and a custom python script was used to design gRNAs as follows: The nucleotide sequence and it's reverse complement were queried for 23 nucleotide sequences that have base C at position 4-8 and bases NGG at position 21-23 where N can be any of the four bases, corresponding to the PAM site requirement for SpCas9 which of AncBE4max. Sequences were further filtered to exclude guides with homopolymer stretches of four or more nucleotides and a GC content of lower than 30%. Only one gRNA per site was selected.

gRNA Cloning

For cloning gRNAs in a parallel manner, gRNA sequence and adjacent bases were ordered as eblocks from IDT, and cloned into the backbone pSB700 mCherry (addgene #64046) using Gibson cloning. Plasmids were cloned in pools of 8 inserts, transformed into 5-alpha competent E. coli (New England Biolabs), and colonies the gRNA insert sequence was analyzed using Sanger sequencing. Sequence-verified gRNA plasmids were mini-prepped (New England Biolabs) for transfection.

### Cell Culture and Transfection

HEK293T cells were obtained from ATCC and were authenticated and tested negative for mycoplasma by the manufacturer. Cells were maintained in Dulbecco's modified Eagle's medium with Glutamax and Sodium pyruvate (Gibco) with 10% Fetal Bovine Serum (Gibco) at 37 degrees Celsius and 5% CO2. Medium was exchanged every 3 days and cells were regularly passaged before reaching ˜80% confluency using TrypLE (Gibco) for dissociation.

For transfection, cells were seeded in 12-well plates 24 h prior, and transfected with Lipofectamine 2000 (Thermofisher) according to the manufacturer's instructions with modifications as outlined below: Cells in each well were transfected with 3 ug base editor DNA and 1 μg of gRNAs and using 5 μL of lipofectamine reagent. When multiple gRNAs were used in one transfection, gRNAs were pooled at equimolar concentrations. After transfection, cells were cultivated for 3 days, washed once with PBS before harvest.

### Amplicon Library Preparation and Sequencing

Genomic DNA was extracted using Zymo DNA extraction kit, and target sites were amplified in separate 25 uL PCR reactions using Kapa HiFi Hotstart readymix according to the manufacturer's instructions with xx ng of genomic DNA as template. Libraries were prepared using NEBNextUltra library prep kit using a size selection step, pooled at equimolar concentration and sequenced on an Illumina MiSeq, using paired end sequencing.

### Amplicon Sequencing Analysis Pipeline

Paired end read fastqs were aligned to the human reference genome hg38 using bowtie2 version 2.3.4.3. The resulting aligned files were analyzed using a custom python script. Base pileup for genomic indices corresponding to the key indices was performed using pysam version 0.18.0, with minimum base quality set to 30. The fraction of edited bases was obtained by dividing the number of edited bases at the index position, i.e. T or As, by the sum of both reference bases, i.e. Cs or Gs, and edited bases.

### Sensitivity/Specificity Experiment

gRNAs were split into two batches of 55 gRNAs each, and HEK293 Ts were transfected with AncBE4max and gRNA batches as described above. Edited sites were analyzed using amplicon sequencing and the analysis pipeline described above. The false positive rate at each editing percentage was calculated as follows: False positive rate=FP/(FP+TN). False negative rate was calculated using the following formula: False negative rate=FN/(FN+TP).

### Simulating False Negative Rates

High coverage sequencing data was downloaded from the SRA database SRX5342252. Fastq files were aligned to the human reference genome hg38 using bowtie2 version 2.4.1. Paired end reads of the bam file were unpaired using a custom python script and treated as single end reads. Single base mutations were inserted using biostar404363 (Lindenbaum, 2015), at a distance of at least 450 bases to other artificial mutations to ensure independence of variant calling decisions. Allele percentages of synthetic mutations ranged from 0.001% to 20% at sites with sequencing coverage from ranges 10× to 5000×. 300 sites were chosen for each coverage level and one modified bam file was generated for each allele percent and coverage combination. Variant calling was performed and the false negative rate was determined, comparing two variant callers; Mutect and Varscan2. Varscan2 was run in somatic mode with the unmodified bam file as a normal. The sensitivity flag-min-var-freq was set to the allele frequency of the mutation and a minimum coverage level of 5× was required to call a variant.

### Evaluating False Positive Rates

Applicants defined false positive cases as bases in the original unmodified bam that were called a variant with an allele percent lower than 30% and a depth of at least 5. The false positive count was divided by the number of bases with sequencing depth of at least 5 to derive the false positive rate. Varscan2 was run with pileup2snp to call variants without a normal file for comparison and the sensitivity flag min-var-freq was set to a range of thresholds from 0.001% to 20% to derive the relationship between false positive rate and variant caller sensitivity.

Mutect2 doesn't have a flag that directly affects its sensitivity so Applicants derived its false positive rate using only the vcf file from running Mutect2 on the unmodified bam file once.

Modeling Adversary Cost with False Negative Data

Assuming a message converted into binary is randomly distributed as 50% 0's and 1's, then any given message can at best reveal half of the still undiscovered key indices. Applicants define the reveal rate as, where FN is the false negative rate and assuming a message converted into binary is randomly distributed as 50% 0's and 1's.

$$Reveal Rate ( R R ) = ( 1 - FN ) / 2$$

Applicants assume that if the adversary can discover 90% of the key indices that key is not viable for further use and a new key has to be generated. Using the reveal rate Applicants can derive how many messages an adversary has to sequence to discover 90% of key indices.

$$\begin{matrix}Crack Threshold ( CT ) = 0.9 \\ m = log ( 1 - CT ) / log ( 1 - R R )\end{matrix}$$

For each combination of allele percent and coverage the variant caller false negative rate can be used to derive how many messages it will take the adversary to crack the key. For calculating the total cost, Applicants assume that an adversary chooses the ideal coverage for a given allele frequency. The final cost is calculated by multiplying the number of required messages times the cost for sequencing at the ideal coverage.

$$adversary Cost = m * WGSCost$$

These calculations were performed with a pipeline developed with python and RStudio.

Modeling Adversary Cost with False Positive Data.

The variant caller false positive rate depends on its sensitivity setting which the adversary controls. Applicants assume the adversary will have to set the sensitivity to at least be able to detect the lowest allele percent base edits. Experimentally the editing range is around 0.1% to 5%.

For each base depending on if it is a key index or not, the number of times it is called a variant over m messages will differ. Bases that aren't key indices would be called a variant at the false positive rate while key index bases will be called at a rate equal to the reveal rate. These form two distinct binomial distributions with their mean at the proportion of times a base would be called a variant and sample size being the number of messages sequenced. The decision of whether a base is a key index or not is a question of which distribution it falls under. Key indices have their mean located at the reveal rate and non key indices have their mean at the false positive rate. The overlap between the two distributions decreases with messages sequenced, and Applicants set a threshold for the allowed overlap between the distributions that would allow the adversary to successfully uncover enough key indices to read or tamper with the message: The number of false positives an adversary could allow should be smaller or equal to the number of bits in the message, i.e. ˜100/3*10\hat{ }9. The threshold for allowable false negatives was set to 0.1, equivalent to the crack threshold defined above. A statistical pipeline was developed in RStudio to calculate how many messages the adversary needs to sequence to distinguish false positives from true key indices.

### Comparing Adversary and Recipient Cost

Assuming it costs $1000 to perform WGS on the human genome with an average coverage of 30× Applicants estimated the cost to perform WGS at 1λ coverage to be ˜$33.

Recipient Cost=2*Amplicon size*number of bits*(WGSCost at 1×coverage/

$$Size of genome ) * ( edited base count needed / Allele Percent )$$

Recipient cost was estimated as above. The estimated cost of sequencing a single base (WGS Cost at 1×coverage/size of genome) is multiplied with the number of total bases needed to be sequenced in order to make a decision on whether a key index encodes a 1 or 0. The total cost is multiplied by 2 for paired end sequencing. To determine the coverage needed to see a base edit at least some number of times Applicants divide that number by the allele percent.

### Stability Experiment

HEK293T cells were transfected with exome targeting gRNAs in batches of 4 gRNAs per transfection, and equal numbers of cells per transfection were pooled 3 days after lipofection. For assessing stability over time, cells were maintained in a 12-well plate and passaged at a ratio of 1:8 every three days. At each passage, parts of the cells were harvested for genomic DNA extraction. For the bottleneck experiments, 50, 100, 500 or 1000 cells were sorted into 12-well plates at day 3 after transfection using a SONY SH800 cell sorter. Cells were cultivated for 14 days and harvested for gRNA extraction.

### Whole Exome Sequencing Library Preparation

Genomic DNA was extracted using Zymo DNA extraction kit. A total of 500 ng of genomic DNA was fragmented using NEBNext Ultra II FS DNA Fragmentation module (NEB) for 20 min and 37 C, and whole genome library preparation was carried out using NEBNext Ultra II DNA Library Prep Kit, with a final PCR amplification step of 13 cycles. Subsequently, exonic regions were enriched using NextGen hybridization capture IDT using 500 ng library DNA as input and following the manufacturer's instructions.

### Variant Calling (Experimental Data) GATK Pre Processing

Fastq files were aligned to the human reference genome hg38 using bowtie2 version 2.3.4.3. Next, bam files were processed following GATK4 best practices. Briefly, after alignment duplicates were removed and base quality scores were recalibrated using picard. Processed bam files were then used as input for Mutect and VarScan and variants were called using a sensitivity threshold of 1% and 0.5% for VarScan.

For determining the number of false positives, called variants with over 25% editing were filtered out, since those were assumed to be cell-line specific SNPs. False positive rate was determined by dividing the number of false positive calls by the number of bases covered by the IDT hybridization panel.

### REFERENCES

Anzalone, Andrew V., Peyton B. Randolph, Jessie R. Davis, Alexander A. Sousa, Luke W. Koblan, Jonathan M. Levy, Peter J. Chen, et al. 2019. “Search-and-Replace Genome Editing without Double-Strand Breaks or Donor DNA.” Nature 576 (7785): 149-57.
Chatterjee, Pranam, Jooyoung Lee, Lisa Nip, Sabrina R. T. Koseki, Emma Tysinger, Erik J. Sontheimer, Joseph M. Jacobson, and Noah Jakimo. 2020. “A Cas9 with PAM Recognition for Adenine Dinucleotides.” Nature Communications 11 (1): 2474.
Choi, Junhong, Wei Chen, Anna Minkina, Florence M. Chardon, Chase C. Suiter, Samuel G. Regalado, Silvia Domcke, et al. 2022. “A Time-Resolved, Multi-Symbol Molecular Recorder via Sequential Genome Editing.” Nature 608 (7921): 98-107.
Church, George M., Yuan Gao, and Sriram Kosuri. 2012. “Next-Generation Digital Information Storage in DNA.” Science 337 (6102): 1628.
Clelland, C. T., V. Risca, and C. Bancroft. 1999. “Hiding Messages in DNA Microdots.” Nature 399 (6736): 533-34.
Farzadfard, Fahim, Nava Gharaei, Yasutomi Higashikuni, Giyoung Jung, Jicong Cao, and Timothy K. Lu. 2019. “Single-Nucleotide-Resolution Computing and Memory in Living Cells.” Molecular Cell 75 (4): 769-80.e4.
Gaudelli, Nicole M., Alexis C. Komor, Holly A. Rees, Michael S. Packer, Ahmed H. Badran, David I. Bryson, and David R. Liu. 2017. “Programmable Base Editing of A·T to G·C in Genomic DNA without DNA Cleavage.” Nature 551 (7681): 464-71.
Kalhor, Reza, Kian Kalhor, Leo Mejia, Kathleen Leeper, Amanda Graveline, Prashant Mali, and George M. Church. 2018. “Developmental Barcoding of Whole Mouse via Homing CRISPR.” Science 361 (6405). doi.org/10.1126/science.aat9804.
Koblan, Luke W., Jordan L. Doman, Christopher Wilson, Jonathan M. Levy, Tristan Tay, Gregory A. Newby, Juan Pablo Maianti, Aditya Raguram, and David R. Liu. 2018. “Improving Cytidine and Adenine Base Editors by Expression Optimization and Ancestral Reconstruction.” Nature Biotechnology 36 (9): 843-46.
Komor, Alexis C., Yongjoo B. Kim, Michael S. Packer, John A. Zuris, and David R. Liu. 2016. “Programmable Editing of a Target Base in Genomic DNA without Double-Stranded DNA Cleavage.” Nature 533 (7603): 420-24.
McKenna, Aaron, Gregory M. Findlay, James A. Gagnon, Marshall S. Horwitz, Alexander F. Schier, and Jay Shendure. 2016. “Whole-Organism Lineage Tracing by Combinatorial and Cumulative Genome Editing.” Science 353 (6298): aaf7907.
Qian, Jason, Zhi-Xiang Lu, Christopher P. Mancuso, Han-Ying Jhuang, Rocío Del Carmen Barajas-Ornelas, Sarah A. Boswell, Fernando H. Ramírez-Guadiana, et al. 2020. “Barcoded Microbial System for High-Resolution Object Provenance.” Science 368 (6495): 1135-40.
Schiroli, Giulia, Anastasia Conti, Samuele Ferrari, Lucrezia Della Volpe, Aurelien Jacob, Luisa Albano, Stefano Beretta, et al. 2019. “Precise Gene Editing Preserves Hematopoietic Stem Cell Function Following Transient p53-Mediated DNA Damage Response.” Cell Stem Cell 24 (4): 551-65.e8.
Shannon, C. E. 1949. “Communication Theory of Secrecy Systems.” The Bell System Technical Journal 28 (4): 656-715.
Shipman, Seth L., Jeff Nivala, Jeffrey D. Macklis, and George M. Church. 2017. “CRISPR-Cas Encoding of a Digital Movie into the Genomes of a Population of Living Bacteria.” Nature 547 (7663): 345-49.
Tang, Weixin, and David R. Liu. 2018. “Rewritable Multi-Event Analog Recording in Bacterial and Mammalian Cells.” Science 360 (6385). doi.org/10.1126/science.aap8992.
Walton, Russell T., Kathleen A. Christie, Madelynn N. Whittaker, and Benjamin P. Kleinstiver. 2020. “Unconstrained Genome Targeting with near-PAMless Engineered CRISPR-Cas9 Variants.” Science. doi.org/10.1126/science.aba8853.
Yim, Sung Sun, Ross M. McBee, Alan M. Song, Yiming Huang, Ravi U. Sheth, and Harris H. Wang. 2021. “Robust Direct Digital-to-Biological Data Storage in Living Cells.” Nature Chemical Biology 17 (3): 246-53.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of encryption comprising:
a. configuring one or more nucleic acid modifying agents to edit a plurality of genomic loci according to one or more encryption keys, wherein the one or more encryption keys link encoded information to a genomic loci thereby creating a set of genomic loci coordinates that hold the encoded information and defining an allele status for each genomic loci in the set of genomic loci coordinates; and
b. editing the plurality of genomic loci by introducing the one or more nucleic acid modifying agents to a cell or population of cells, whereby information is encrypted within one or more genomes of the cell or population of cells.

2. The method of claim 1, further comprising decoding the information by observing an allele frequency at the genomic loci defined by the one or more encryption keys.

3. The method of claim 2, wherein observing the allele frequency comprises amplifying the plurality of genomic loci defined by the one or more encryption keys and sequencing the amplified genomic loci to determine the allele frequency.

4. The method of any of claims 1-3, wherein the encoded information comprises digital or biological data.

5. The method of claim 2, wherein the encoded information further includes an authentication code defining an expected allele frequency, and wherein the decoding step further comprises comparing the expected allele frequency to an observed allele frequency, wherein an increase in the observed allele frequency relative to the expected allele frequency indicates inauthentic or invalid information.

6. The method of any one of the preceding claims, wherein the encoded information is binary encoded.

7. The method of claim 4, wherein an edited genomic locus corresponds to a first binary value and a non-edited genomic loci corresponds to a second binary value.

8. The method of any one of the preceding claims, wherein the allelic frequency of the alleles to the one or more genomes is less than 10%, less than 5%, less than 3%, less than 2%, less than 1%, less than 0.5%, or less than 0.1%.

9. The method of any one of the preceding claims, wherein the one or more nucleic acid modifying agents are configured to edit the plurality of genomic loci according to one or more chaff edits.

10. The method of claim 9, wherein the one or more chaff edits are interspersed among the genomic loci according to the one or more encryption keys.

11. The method of claim 9 or 10, wherein the one or more chaff edits are randomly assigned.

12. The method of any of the preceding claims, wherein the encoded information is encrypted in a set of key genomic loci coordinates, the key genomic loci coordinates being a subset of the genomic loci coordinates.

13. The method of any one of the preceding claims, wherein an order of the genomic loci is randomized.

14. The method of any one of the preceding claims, wherein multiple edits are carried out in parallel.

15. The method of any one of the preceding claims, wherein the edit comprises changing a single nucleobase to another nucleobase.

16. The method of any of the preceding claims, wherein the one or more nucleic acid modifying agent is a base editing system or a prime editing system.

17. The method of claim 16, wherein the base editing system comprises a cytidine deaminase or an adenosine deaminase.

18. The method of claim 16 or 17, wherein the base editing system is engineered to have a relaxed PAM requirement, multiple base editing systems having different PAM requirements are used, the base editing system is used with another nucleic acid modifying agent that has no PAM requirement or a different PAM requirement, or a combination thereof.

19. The method of claim 15, wherein the nucleic acid modifying agent is a CRISPR-Cas, Zn Finger nuclease, a TALEN, or an Omega System that directs insertion of the edit via homology directed repair and a donor template comprising one or more edits.

20. The method of claim 15, wherein the nucleic acid modifying agent is a CRISPR-associated transposase (CAST) system that directs insertion of the edit via transposase-mediated insertion of a donor template comprising one or more edits.

21. The method of any of the preceding claims, wherein the one or more genomes are from a prokaryote, a eukaryote, or a combination thereof.

22. An engineered, non-naturally occurring cell, or progeny thereof, wherein the genome of the cell is modified to store encoded information encrypted according to the method of any of claims 1 to 21.

23. A method of encoding an authentication signature into a biological material, comprising encoding an encrypted verification signature in one or more genomes of the biological material by introducing edits using one or nucleic acid modifying agents at a plurality of genomic loci defined according to one or more encryption keys, whereby measuring the plurality of the genomic loci as defined by the one or more encryption keys can be used to identify and/or authenticate an origin or source of the biological material.

24. A method of authenticating a biological material, comprising adding one or more cells to the biological material, the one or more cells comprising information encrypted in a genome(s) of the one or more cells, wherein the encrypted information is used to authenticate the biological material.

25. The method of claim 24, wherein the one or more cells are the engineered cells of claim 22.

26. A method of authenticating a biological material comprising:
measuring a set of genomic loci from one or more cells obtained from the biological material and as defined by one or more encryption keys, wherein at least a portion of the cells of the biological material comprises genomes previously edited with one or more nucleic acid modifying agents to encode an authentication signature according to the one or more encryption keys;
wherein an observed allele status at the genomic loci, in combination with the one or more encryption keys, are used to decode the authentications signature that confirms an identity of and/or authenticates an origin of the biological material.

27. The method of claim 23 or 26, further comprising decoding the authentication signature by observing an allele frequency at the genomic loci defined by the one or more encryption keys.

28. The method of claim 27, wherein observing the allele frequency comprises amplifying the plurality of genomic loci defined by the one or more encryption keys and sequencing the amplified genomic loci to determine the allele frequency.

29. The method of claim 23 or 26, wherein measuring comprises performing an allele detection method.

30. The method of claim 23 or 26, wherein the biological material is a modified organism or a modified cell.

31. The method of claim 30, wherein the modified organism is a modified plant.

32. The method of claim 30, wherein the modified cell is a therapeutic cell.

33. The method of claim 23 or 26, wherein the authentication signature further includes an authentication code defining an expected allele frequency, and wherein the decoding step further comprises comparing the expected allele frequency to an observed allele frequency, wherein an increase in the observed allele frequency relative to the expected allele frequency indicates inauthentic or invalid information.

34. The method of any one of claims 23 to 33, wherein the authentication signature is binary encoded.

35. The method of claim 34, wherein an edited genomic locus corresponds to a first binary value and a non-edited genomic loci corresponds to a second binary value.

36. The method of any one of claim 33, wherein the expected allele frequency of the edits to the one or more genomes is less than 10%, less than 5%, less than 3%, less than 2%, less than 1%, less than 0.5%, or less than 0.1%.

37. The method of any one of claims 23 to 36, wherein the one or more nucleic acid modifying agents are configured to edit the plurality of genomic loci according to one or more chaff edits.

38. The method of claim 37, wherein the one or more chaff edits are interspersed among the genomic loci according to the one or more encryption keys.

39. The method of claim 37 or 38, wherein the one or more chaff edits are randomly assigned.

40. The method of any one of claims 23 to 37, wherein an order of the genomic loci is randomized.

41. The method of any one of claims 23 to 40, wherein the nucleic acid modifying agent is a Zn Finger nuclease, a TALEN, a meganuclease, a CRISPR-Cas system, a CAST system, ARCUS, a base editing system, a prime editing system, or a combination thereof.

42. The method of claim 41, wherein the nucleic acid modifying agent is a base editing system or a prime editing system.

43. The method of claim 42, wherein the base editing system comprises a cytidine deaminase or an adenosine deaminase.
